(19) United States
(12) Patent Application Publication
(10) Pub. No.: US 2009/0214527 A1
(43) Pub. Date: Aug. 27, 2009

(54) Combination Of Anti-Madcam Antibody And Antifibrotic Caspase Inhibitor To Treat Liver Fibrosis

(76) Inventors: Gary Burgess, Kent (GB); Roger Kerry, Kent (GB)

Correspondence Address: PFIZER INC.;PATENT DEPARTMENT, Bld 114 M/S 114, EASTERN POINT ROAD, GROTON, CT, 06340, US

(21) Appl. No.: 11/995,204

(22) PCT Filed: Jul. 3, 2006
(86) PCT No.: PCT/IB06/01896

§ 371 Date: Jan. 10, 2008

Related U.S. Application Data
(60) Provisional application No. 60/698,561, filed on Jul. 11, 2005.

(51) Int. Cl. A61K 39/395; A61P 1/16

(52) U.S. Cl. 424/133.1; 424/144.1; 424/172.1

(57) ABSTRACT

The invention relates to a new combination of an anti-MAdCAM antibody with an anti-fibrotic agent, such as a protease inhibitor, preferably a caspase inhibitor. The invention also relates to pharmaceutical compositions comprising the combination of the invention, and the use of the combination for the treatment of liver fibrosis.

This application is a national filing of PCT/IB06/001896 filed Jul. 3, 2006 which claims priority to U.S. Provisional Application No. 60/698,561, filed Jul. 11, 2005.

The invention relates to a new combination of an anti-MAdCAM antibody with an anti-fibrotic agent, such as a protease inhibitor, preferably a caspase inhibitor. The invention also relates to pharmaceutical compositions comprising the combination of the invention, and the use of the combination for the treatment of liver fibrosis.

Chronic liver disease is responsible for over 1.4 m deaths annually (WHO, World Health Report 2004) and in the US is among the top ten disease related causes of death (CDC, National Center for Health Statistics, 2004). Liver fibrosis, which is an outcome of persistent hepatic inflammation, if left unmanaged has serious long-term consequences for patient morbidity and mortality.

Liver fibrosis is one of the processes that occurs when the liver is damaged. Such damage may be the result of, for example, viral activity (e.g., chronic hepatitis types B or C) or other liver infections (e.g., parasites, bacteria); chemicals (e.g., pharmaceuticals, recreational drugs, excessive alcohol, exposure to pollutants); immune processes (e.g., autoimmune hepatitis); metabolic disorders (e.g., lipid, glycogen, or metal storage disorders); or cancer growth (primary or secondary liver cancer). Fibrosis is both a sign of liver damage and a potential contributor to liver failure via progressive cirrhosis of the liver.

There is a serious unmet medical need. The present invention relate to a combination of an anti-mucosal addressin cell adhesion molecule (MAdCAM) antibody with an anti-fibrotic agent, preferably a protease inhibitor, more preferably a caspase inhibitor, which is beneficial for the treatment of liver fibrosis.

Mucosal addressin cell adhesion molecule (MAdCAM) is a member of the immunoglobulin superfamily of cell adhesion receptors. It is one of the adhesion molecules involved in the recruitment of lymphocytes o tissues when required, by means of interacting with an integrin molecule on the surface of the lymphocytes.

It has been shown that antibodies that inhibit binding of MAdCAM to its integrin binding partner, α₄β₇, for example anti-MAdCAM antibodies (e.g. MECA-367; U.S. Pat. No. 5,403,919, U.S. Pat. No. 5,538,724) or anti-α₄β₇ antibodies (e.g. Act-1; U.S. Pat. No. 6,551,593), can inhibit leukocyte extravasation into inflamed intestine, and can therefore be beneficial in the treatment of inflammatory bowel disease (IBD).

### ASPECTS OF THE INVENTION

One aspect of the invention is a combination of an anti-MAdCAM antibody or antigen-binding portion thereof with an anti-fibrotic agent. Preferably, the anti-fibrotic agent is a protease inhibitor, more preferably a caspase inhibitor, even more preferably the caspase inhibitor is a compound of formula I:

The compound of formula I

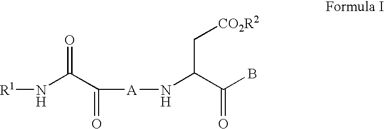

wherein A, B, R1 and R2 are as defined below.

Preferably, the compound of formula I is selected from:
(3S)-3-[N—(N′-(2-Fluoro-4-Iodophenyl)Oxamyl)Valinyl]Amino-5-(2′,3′,5′,6′-Tetrafluorophenoxy)-4-Oxopentanoic acid;
(3S)-3-[N—(N′-(2-Chlorophenyl)Oxamyl)Valinyl]Amino-5-(2′,3′,5′,6′-Tetrafluorophenoxy)-4-Oxopentanoic acid;
(3S)-3-[N—(N′-(2-Bromophenyl)Oxamyl)Valinyl]Amino-5-(2′,3′,5′,6′-Tetrafluorophenoxy)-4-Oxopentanoic acid;
(3S)-3-[N—(N′-(2-Fluorophenyl)Oxamyl)Valinyl]Amino-5-(2′,3′,5′,6′-Tetrafluorophenoxy)-4-Oxopentanoic acid;
(3S)-3-[N—(N′-(2-Trifluoromethylphenyl)Oxamyl)Valinyl]Amino-5-(2′,3′,5′,6′-Tetrafluorophenoxy)-4-Oxopentanoic acid;
(3S)-3-[N—(N′-(1-Anthryl)Oxamyl)Valinyl] Amino-5-(2′,3′,5′,6′-Tetrafluorophenoxy)-4 Oxopentanoic acid;
(3S)-3-[N—(N′-(2-Tert-Butylphenyl)Oxamyl)Alaninyl]Amino-5-(2′,3′,5′,6′-Tetrafluorophenoxy)-4-Oxopentanoic acid;
(3S)-3-[N—(N′-(2-Trifluoromethylphenyl)Oxamyl)Alaninyl]Amino-5-(2′,3′,5′,6′-Tetrafluorophenoxy)-4-Oxopentanoic acid;
(3S)-3-[N—(N′-(2,6-Difluorophenyl)Oxamyl)Alaninyl]Amino-5-(2′,3′,5′,6′-Tetrafluorophenoxy)-4-Oxopentanoic acid;
(3S)-3-[N—(N′-(1-Naphthyl)Oxamyl)Alaninyl]Amino-5-(2′,3′,5′,6′-Tetrafluorophenoxy)-4-Oxopentanoic acid;
(3S)-3-[N—(N′-(4-Methoxyphenyl)Oxamyl)Alaninyl]Amino-5-(2′,3′,5′,6′-Tetrafluorophenoxy)-4-Oxopentanoic acid;
(3S)-3-[N—(N′-(2-Trifluoromethylphenyl)Oxamyl)Valinyl]Amino-4-Oxobutanoic acid;
(3S)-3-[N—(N′-(2-tert-Butylmethyl phenyl)Oxamyl)Valinyl]Amino-4-Oxobutanoic acid;
(3S)-3-[N—(N′-(2-Benzylphenyl)Oxamyl)Valinyl]Amino-4-Oxobutanoic acid;
(3S)-3-[N—(N′-(2-Phenylphenyl)Oxamyl)Valinyl]Amino-4-Oxobutanoic acid.

Even more preferably, the compound is (3S)-3-[N—(N′-(2-Tert-Butylphenyl)Oxamyl)Alaninyl]Amino-5-(2′,3′,5′,6′-Tetrafluorophenoxy)-4-Oxopentanoic acid.

In one aspect of the invention the anti-MAdCAM antibody or antigen-binding portion thereof in the combination is an antibody or antigen-binding portion thereof that specifically binds MAdCAM. Preferably, the antibody or portion is a human monoclonal antibody or antigen-binding portion thereof. Preferably, the antibody or portion possesses at least one of the following properties:

(a) binds to human cells;
(b) has a selectivity for MAdCAM over VCAM or fibronectin of at least 100 fold;
(c) binds to human MAdCAM with a Kd of 3×10-¹⁰ M or less; or
(d) inhibits the binding of α₄β₇ expressing cells to human MAdCAM.
(e) inhibits the recruitment of lymphocytes to gastrointestinal lymphoid tissue.

Preferably, the antibody or antigen-binding portion inhibits binding of human MAdCAM to α₄β₇, and has at least one of the following properties:

(a) cross-competes with a reference antibody for binding to MAdCAM;
(b) competes with a reference antibody for binding to MAdCAM;
(c) binds to the same epitope of MAdCAM as a reference antibody;
(d) binds to MAdCAM with substantially the same K_{d} as a reference antibody;
(e) binds to MAdCAM with substantially the same off rate as a reference antibody;
wherein the reference antibody is selected from the group consisting of: monoclonal antibody 1.7.2, monoclonal antibody 1.8.2, monoclonal antibody 6.14.2, monoclonal antibody 6.22.2, monoclonal antibody 6.34.2, monoclonal antibody 6.67.1, monoclonal antibody 6.73.2, monoclonal antibody 6.77.1, monoclonal antibody 7.16.6, monoclonal antibody 7.20.5, monoclonal antibody 7.26.4, monoclonal antibody 9.8.2, monoclonal antibody 6.22.2-mod, monoclonal antibody 6.34.2-mod, monoclonal antibody 6.67.1-mod, monoclonal antibody 6.77.1-mod and monoclonal antibody 7.26.4-mod.

In another aspect of the invention the heavy chain variable region, the light chain variable region or both of the anti-MAdCAM antibody are at least 90% identical in amino acid sequence to the corresponding region or regions of a monoclonal antibody selected from the group consisting of: monoclonal antibody 1.7.2, monoclonal antibody 1.8.2, monoclonal antibody 6.14.2, monoclonal antibody 6.22.2, monoclonal antibody 6.34.2, monoclonal antibody 6.67.1, monoclonal antibody 6.73.2, monoclonal antibody 6.77.1, monoclonal antibody 7.16.6, monoclonal antibody 7.20.5, monoclonal antibody 7.26.4 monoclonal antibody 9.8.2, monoclonal antibody 6.22.2-mod, monoclonal antibody 6.34.2-mod, monoclonal antibody 6.67.1-mod, monoclonal antibody 6.77.1-mod and monoclonal antibody 7.26.4-mod.

Preferably the antibody is selected from the group consisting of:

(a) an antibody comprising the amino acid sequences set forth in SEQ ID NO: 2 and SEQ ID NO: 4, without the signal sequences;
(b) an antibody comprising the amino acid sequences set forth in SEQ ID NO: 6 and SEQ ID NO: 8, without the signal sequences;
(c) an antibody comprising the amino acid sequences set forth in SEQ ID NO: 10 and SEQ ID NO: 12, without the signal sequences;
(d) an antibody comprising the amino acid sequences set forth in SEQ ID NO: 14 and SEQ ID NO: 16, without the signal sequences;
(e) an antibody comprising the amino acid sequences set forth in SEQ ID NO: 18 and SEQ ID NO: 20, without the signal sequences;
(f) an antibody comprising the amino acid sequences set forth in SEQ ID NO: 22 and SEQ ID NO: 24, without the signal sequences;
(g) an antibody comprising the amino acid sequences set forth in SEQ ID NO: 26 and SEQ ID NO: 28, without the signal sequences;
(h) an antibody comprising the amino acid sequences set forth in SEQ ID NO: 30 and SEQ ID NO: 32, without the signal sequences;
(i) an antibody comprising the amino acid sequences set forth in SEQ ID NO: 34 and SEQ ID NO: 36, without the signal sequences;
(j) an antibody comprising the amino acid sequences set forth in SEQ ID NO: 38 and SEQ ID NO: 40, without the signal sequences;
(k) an antibody comprising the amino acid sequences set forth in SEQ ID NO: 42 and SEQ ID NO: 44, without the signal sequences;
(l) an antibody comprising the amino acid sequences set forth in SEQ ID NO: 46 and SEQ ID NO: 48, without the signal sequences;
(m) an antibody comprising the amino acid sequences set forth in SEQ ID NO: 52 and SEQ ID NO: 54, without the signal sequences;
(n) an antibody comprising the amino acid sequences set forth in SEQ ID NO: 56 and SEQ ID NO: 58, without the signal sequences;
(o) an antibody comprising the amino acid sequences set forth in SEQ ID NO: 60 and SEQ ID NO: 62, without the signal sequences;
(p) an antibody comprising the amino acid sequences set forth in SEQ ID NO: 64 and SEQ ID NO: 66, without the signal sequences; and
(q) an antibody comprising the amino acid sequences set forth in SEQ ID NO: 42 and SEQ ID NO: 68, without the signal sequences.

In one aspect of the invention, the heavy chain C-terminal lysine may be cleaved from the anti-MAdCAM antibody of the combination.

In another aspect of the invention, the monoclonal antibody or an antigen-binding portion thereof is selected from the following antibodies:

(a) the heavy chain comprises the heavy chain CDR1, CDR2 and CDR3 amino acid sequences of a reference antibody selected from the group consisting of: 1.7.2, 1.8.2, 6.14.2, 6.22.2, 6.34.2, 6.67.1, 6.73.2, 6.77.1, 7.16.6, 7.20.5, 7.26.4, 9.8.2, 6.22.2-mod, 6.34.2-mod, 6.67.1-mod, 6.77.1-mod and 7.26.4-mod
(b) the light chain comprises the light chain CDR1, CDR2 and CDR3 amino acid sequences of a reference antibody selected from the group consisting of: 1.7.2, 1.8.2, 6.14.2, 6.22.2, 6.34.2, 6.67.1, 6.73.2, 6.77.1, 7.16.6, 7.20.5, 7.26.4, 9.8.2, 6.22.2-mod, 6.34.2-mod, 6.67.1-mod, 6.77.1-mod and 7.26.4-mod
(c) the antibody comprises a heavy chain of (a) and a light chain of (b); and
(d) the antibody of (c) wherein the heavy chain and light chain CDR amino acid sequences are selected from the same reference antibody.

In another aspect of the invention, the monoclonal antibody or antigen-binding portion comprises:

(a) a heavy chain comprising the heavy chain variable region amino acid sequence of an antibody selected from the group consisting of: 1.7.2 (SEQ ID NO: 2); 1.8.2 (SEQ ID NO: 6); 6.14.2 (SEQ ID NO: 10); 6.22.2 (SEQ ID NO: 14); 6.34.2 (SEQ ID NO: 18); 6.67.1 (SEQ ID NO: 22); 6.73.2 (SEQ ID NO: 26); 6.77.1 (SEQ ID NO: 30); 7.16.6 (SEQ ID NO: 34); 7.20.5 (SEQ ID NO: 38); 7.26.4 (SEQ ID NO: 42); and 9.8.2 (SEQ ID NO: 46); 6.22.2-mod (SEQ ID NO: 52); 6.34.2-mod (SEQ ID NO: 56); 6.67.1-mod (SEQ ID NO: 60); 6.77.1-mod (SEQ ID NO: 64); and 7.26.4-mod (SEQ ID NO: 42);
(b) a light chain comprising the light chain variable region amino acid sequence of an antibody selected from the group consisting of: 1.7.2 (SEQ ID NO: 4); 1.8.2 (SEQ ID NO: 8); 6.14.2 (SEQ ID NO: 12); 6.22.2 (SEQ ID NO: 16); 6.34.2 (SEQ ID NO: 20); 6.67.1 (SEQ ID NO: 24); 6.73.2 (SEQ ID NO: 28); 6.77.1 (SEQ ID NO: 32); 7.16.6 (SEQ ID NO: 36); 7.20.5 (SEQ ID NO: 40); 7.26.4 (SEQ ID NO: 44); and 9.8.2 (SEQ ID NO: 48); 6.22.2-mod (SEQ ID NO: 54); 6.34.2-mod (SEQ ID NO: 58); 6.67.1-mod (SEQ ID NO: 62); 6.77.1-mod (SEQ ID NO: 66); and 7.26.4-mod (SEQ ID NO: 68); or
(c) the heavy chain of (a) and the light chain of (b).

Yet another aspect of the invention is a combination of an anti-α₄β₇ integrin antibody or antigen binding portion thereof with a caspase inhibitor as defined herein. Preferably, the antibody inhibits the interaction of MAdCAM with α₄β₇ integrin. More preferably, the antibody is humanised Act-1, also called MLN02 (WO 01/078779).

Another aspect of the invention is the medical use of any combination described herein, preferably the use for the treatment of liver fibrosis, including but not limited to, the treatment of Hepatitis C-induced liver damage, alcoholic liver disease, non-alcoholic steatohepatitis (NASH).

Another aspect of the invention is a method of treatment of liver fibrosis including but not limited to, the treatment of Hepatitis C-induced liver damage, alcoholic liver disease, non-alcoholic steatohepatitis (NASH), using an anti-MAdCAM antibody as described herein and an anti-fibrotic agent as described herein.

Another aspect of the invention is a pharmaceutical composition comprising any combination described herein with a pharmaceutically acceptable carrier or excipient.

Another aspect of the invention is a pharmaceutical product comprising an anti-MAdCAM antibody as described herein and an anti-fibrotic agent as described herein for simultaneous, separate, or sequential use.

A further aspect of the invention is a kit comprising an anti-MAdCAM antibody as described herein and an anti-fibrotic agent as described herein.

### DETAILED DESCRIPTION OF THE INVENTION

The anti-fibrotic agent in combination of the invention is preferably a protease inhibitor, more preferably a caspase inhibitor, and even more preferably a compound of formula I

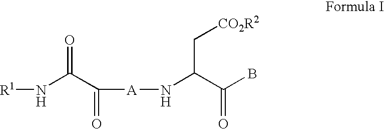

wherein
A is a natural or unnatural amino acid of Formula IIa-i:

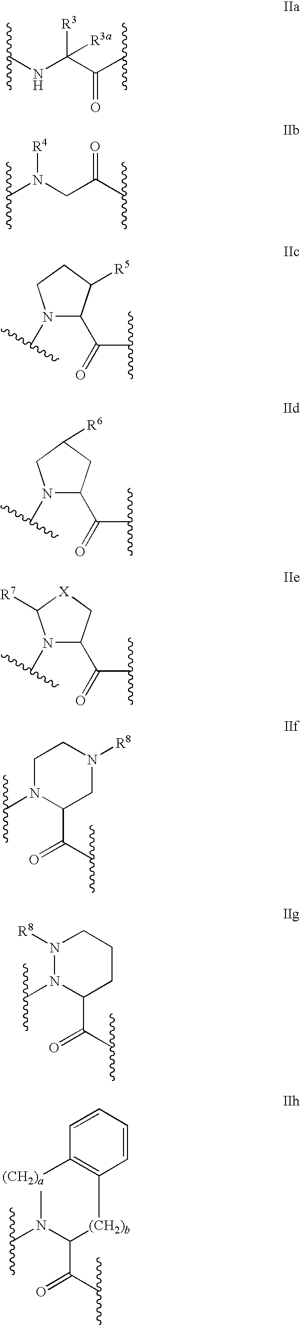

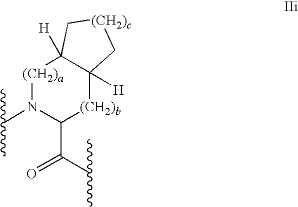

B is a hydrogen atom, a deuterium atom, alkyl, cycloalkyl, phenyl, substituted phenyl, naphthyl, substituted naphthyl, 2-benzoxazolyl, substituted 2-oxazolyl, (CH₂)ₙ-cycloalkyl, (CH₂)ₙ-phenyl, (CH₂)ₙ-(substituted phenyl), (CH₂)ₙ-(1 or 2-naphthyl), (CH₂)ₙ-(substituted 1 or 2-naphthyl), (CH₂)ₙ-(heteroaryl), (CH₂)ₙ-(substituted heteroaryl), halomethyl, CO₂R¹², CONR³R⁴, CH₂ZR¹⁵, CH₂OCO(aryl), CH₂OCO(heteroaryl), or CH₂OPO(R¹⁶)R¹⁷, where Z is an oxygen or a sulfur atom, or B is a group of the Formula IIIa-c:

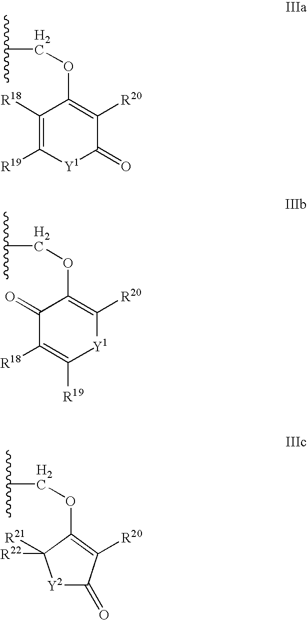

R¹ is alkyl, cycloalkyl, (cycloalkyl)alkyl, phenyl, substituted phenyl, phenylalkyl, substituted phenylalkyl, naphthyl, substituted naphthyl, (1 or 2 naphthyl)alkyl, substituted (1 or 2 naphthyl)alkyl, heteroaryl, substituted heteroaryl, (heteroaryl)alkyl, substituted (heteroaryl)alkyl, R^{1a}(R^{1b})N, or R^{1c}O; and
R² is hydrogen, lower alkyl, cycloalkyl, (cycloalkyl)alkyl, phenyl, substituted phenyl, phenylalkyl, substituted phenylalkyl, naphthyl, substituted naphthyl, (1 or 2 naphthyl)alkyl, or substituted (1 or 2 naphthyl)alkyl;
And wherein:
R^{1a} and R^{1b} are independently hydrogen, alkyl, cycloalkyl, (cycloalkyl)alkyl, phenyl, substituted phenyl, phenylalkyl, substituted phenylalkyl, naphthyl, substituted naphthyl, (1 or 2 naphthyl)alkyl, substituted (1 or 2 naphthyl)alkyl, heteroaryl, substituted heteroaryl, (heteroaryl)alkyl, or substituted (heteroaryl)alkyl, with the proviso that R^{1a} and R^{1b} cannot both be hydrogen;
R^{1c} is alkyl, cycloalkyl, (cycloalkyl)alkyl, phenyl, substituted phenyl, phenylalkyl, substituted phenylalkyl, naphthyl, substituted naphthyl, (1 or 2 naphthyl)alkyl, substituted (1 or 2 naphthyl)alkyl, heteroaryl, substituted heteroaryl, (heteroaryl)alkyl, or substituted (heteroaryl)alkyl;
R³ is C₁₋₆ alkyl, cycloalkyl, phenyl, substituted phenyl, (CH₂)ₙNH₂, (CH₂)NHCOR⁹, (CH₂)ₙN(C═NH)NH₂, (CH₂)ₘCO₂R², (CH₂)ₘOR¹⁰, (CH₂)ₘSR¹¹, (CH₂)ₙcycloalkyl, (CH₂)ₙphenyl, (CH₂)ₙ(substituted phenyl), (CH₂)ₙ(1 or 2-naphthyl) or (CH₂)ₙ(heteroaryl), wherein heteroaryl includes pyridyl, thienyl, furyl, thiazolyl, imidazolyl, pyrazolyl, isoxazolyl, pyrazinyl, pyrimidyl, triazinyl, tetrazolyl, and indolyl;
R^{3a} is hydrogen or methyl, or R³ and R^{3a} taken together are —(CH₂)_{d}— where d is an integer from 2 to 6;
R⁴ is phenyl, substituted phenyl, (CH₂)ₘphenyl, (CH₂)ₘ(substituted phenyl), cycloalkyl, or benzofused cycloalkyl;
R⁵ is hydrogen, lower alkyl, cycloalkyl, phenyl, substituted phenyl, (CH₂)ₙcycloalkyl, (CH₂)ₙphenyl, (CH₂)ₙ(substituted phenyl), or (CH₂)ₙ(1 or 2-naphthyl);
R⁶ is hydrogen, fluorine, oxo, lower alkyl, cycloalkyl, phenyl, substituted phenyl, naphthyl, (CH₂)ₙcycloalkyl, (CH₂)ₙphenyl, (CH₂)ₙ(substituted phenyl), (CH₂)ₙ(1 or 2-naphthyl), OR¹⁰, SR¹¹, or NHCOR⁹;
R⁷ is hydrogen, oxo (i.e. ═O), lower alkyl, cycloalkyl, phenyl, substituted phenyl, naphthyl, (CH₂)ₙcycloalkyl, (CH₂)ₙphenyl, (CH₂)ₙ(substituted phenyl), or (CH₂)ₙ(1 or 2-naphthyl);
R⁸ is lower alkyl, cycloalkyl, (CH₂)ₙcycloalkyl, (CH₂)ₙphenyl, (CH₂)ₙ(substituted phenyl), (CH₂)ₙ(1 or 2-naphthyl), or COR⁹;
R⁹ is hydrogen, lower alkyl, cycloalkyl, phenyl, substituted phenyl, naphthyl, (CH₂)ₙcycloalkyl, (CH₂)ₙphenyl, (CH₂)ₙ(substituted phenyl), (CH₂)ₙ(1 or 2-naphthyl), OR¹², or NR¹³R¹⁴;
R¹⁰ is hydrogen, lower alkyl, cycloalkyl, phenyl, substituted phenyl, naphthyl, (CH₂)ₙcycloalkyl, (CH₂)ₙphenyl, (CH₂)ₙ(substituted phenyl), or (CH₂)ₙ(1 or 2-naphthyl);
R¹¹ is lower alkyl, cycloalkyl, phenyl, substituted phenyl, naphthyl, (CH₂)ₙcycloalkyl, (CH₂)ₙphenyl, (CH₂)ₙ(substituted phenyl), or (CH₂)ₙ(1 or 2-naphthyl);
R¹² is lower alkyl, cycloalkyl, (CH₂)ₙcycloalkyl, (CH₂)ₙphenyl, (CH₂)ₙ(substituted phenyl), or (CH₂)ₙ(1 or 2-naphthyl);
R¹³ is hydrogen, lower alkyl, cycloalkyl, phenyl, substituted phenyl, naphthyl, substituted naphthyl, (CH₂)ₙcycloalkyl, (CH₂)ₙphenyl, (CH₂)ₙ(substituted phenyl), or (CH₂)ₙ(1 or 2-naphthyl);
R¹⁴ is hydrogen or lower alkyl; or
R¹³ and R¹⁴ taken together form a five to seven membered carbocyclic or heterocyclic ring, such as morpholine, or N-substituted piperazine;
R¹⁵ is phenyl, substituted phenyl, naphthyl, substituted naphthyl, heteroaryl, (CH₂)ₙphenyl, (CH₂)ₙ(substituted phenyl), (CH₂)ₙ(1 or 2-naphthyl), or (CH₂)ₙ(heteroaryl);
R¹⁶ or R¹⁷ are independently lower alkyl, cycloalkyl, phenyl, substituted phenyl, naphthyl, phenylalkyl, substituted phenylalkyl, or (cycloalkyl)alkyl;
R¹⁸ and R¹⁹ are independently hydrogen, alkyl, phenyl, substituted phenyl, (CH₂)ₙphenyl, (CH₂)ₙ(substituted phenyl), or R¹⁸ and R¹⁹ taken together are —(CH═CH)₂;
R²⁰ is hydrogen, alkyl, phenyl, substituted phenyl, (CH₂)ₙphenyl, (CH₂)ₙ(substituted phenyl);
R²¹, R²² and R²³ are independently hydrogen, or alkyl;

### X is CH₂, (CH₂)₂, (CH₂)₃, or S;

### Y¹ is O or NR²³;

### Y² is CH₂, O, or NR²³;

a is 0 or 1 and b is 1 or 2, provided that when a is 1 then b is 1;
c is 1 or 2, provided that when c is 1 then a is 0 and b is 1;
m is 1 or 2; and
n is 1, 2, 3, or 4;
or a pharmaceutically acceptable salt thereof.

As used herein, the term “alkyl” means a straight or branched C₁ to C₁₀ carbon chain, such as methyl, ethyl, tert-butyl, iso-propyl, n-octyl, and the like. The term “lower alkyl” means a straight chain or branched C₁ to C₆ carbon chain, such as methyl, ethyl, iso-propyl, and the like.

The term “cycloalkyl” means a mono-, bi-, or tricyclic ring that is either fully saturated or partially unsaturated. Examples of such a ring include cyclopropyl, cyclobutyl, cyclopentyl, cyclohexyl, cycloheptyl, adamantyl, cyclooctyl, cis or trans decalin, bicyclo[2.2.1]hept-2-ene, cyclohex-1-enyl, cyclopent-1-enyl, 1,4-cyclooctadienyl, and the like.

The term “(cycloalkyl)alkyl” means the above-defined alkyl group substituted with one for the above cycloalkyl rings. Examples of such a group include (cyclohexyl)methyl, 3-(cyclopropyl)-n-propyl, 5-(cyclopentyl)hexyl, 6-adamantyl)hexyl, and the like.

The term “substituted phenyl” specifies a phenyl group substituted with one or more substituents chosen from halogen, hydroxy, protected hydroxy, cyano, nitro, trifluoromethyl, alkyl, alkoxy, acyl, acyloxy, carboxy, protected carboxy, carboxymethyl, protected carboxymethyl, hydroxymethyl, protected hydroxymethyl, amino, protected amino, (monosubstituted)amino, protected (monosubstituted)amino, (disubstituted)amino, carboxamide, protected carboxamide, N-(lower alkyl)carboxamide, N-((lower alkyl)sulfonyl)amino, N-(phenylsulfonyl)amino or by a substituted or unsubstituted phenyl group, such that in the latter case a biphenyl or naphthyl group results, or wherein two adjacent alkyl substituents on the substituted phenyl ring taken together form a cycloalkyl to yield, for example, tetrahydronaphthyl or indanyl.

Examples of the term “substituted phenyl” includes a mono-, di-, tri-, tetra, or penta(halo)phenyl group such as 2-, 3-, or 4-chlorophenyl, 2,6-dichlorophenyl, 2,5-dichlorophenyl, 3,4-dichlorophenyl, 2,3- or 4-bromophenyl, 3,4-dibromophenyl, 3-chloro-4-fluorophenyl, 2-, 3- or 4-fluorophenyl, 2,4,6-trifluorophenyl, 2,3,5,6-tetrefluorophenyl, 2,3,4,5-tetrafluorophenyl, 2,3,4,5,6-pentafluorophenyl, and the like; a mono or di(hydroxy)phenyl group such as 2-, 3-, or 4-hydroxyphenyl, 2,4-dihydroxyphenyl, the protected hydroxy derivatives thereof and the like; a nitrophenyl group such as 2-, 3-, or 4-nitrophenyl; a cyanophenyl group, for example, 2-, 3-, or 4-cyanophenyl; a mono or di(alkyl)phenyl group such as 2-, 3-, or 4-ethylphenyl, 2-, 3-, or 4-(n-propyl)phenyl and the like; a mono or di(alkoxy)phenyl group, for example, 2,6-dimethoxyphenyl, 2-, 3-, or 4-(iso-propoxy)phenyl, 2-, 3-, or 4-(t-butoxy)phenyl, 3-ethoxy-4-methoxyphenyl and the like; 2-, 3-, or 4-(protected hydroxymethyl)phenyl or 3,4-di(hydroxymethyl)phenyl; a mono- or di(aminomethyl)phenyl or (protected aminomethyl)phenyl such as 2-, 3- or 4-(aminomethyl)phenyl or 2,4-(protected aminomethyl)phenyl; or a mono- or di(N-(methylsulfonylamino))phenyl such as 2-, 3-, or 4-(N-(methylsulfonylamino))phenyl. Also the term “substituted phenyl” represents disubstituted phenyl groups wherein the substituents are different, for example, 3-methyl-4-hydroxyphenyl, 3-hydroxy-4-nitrophenyl, 2-hydroxy-4-chlorophenyl, and the like.

The term “phenylalkyl” means one of the above phenyl groups attached to one of the above-described alkyl groups, and the term “substituted phenylalkyl” means that either the phenyl or the alkyl or both are substituted with one or more of the above-defined substituents. Examples of such groups include 2-phenyl-1-chloroethyl, 2-(4′-methoxyphenyl)ethyl, 4-(2′6′-dihydroxy phenyl)ₙ-hexyl, 2-(5′cyano-3′-methoxyphenyl)ₙ-pentyl, 3-(2′6′-dimethylphenyl)ₙ-propyl, 4-chloro-3-aminobenzyl, 6-(4′-methoxyphenyl)-3-carboxy(n-hexyl), 5-(4′-aminomethylphenyl)-3-(aminomethyl)ₙ-pentyl, 5-phenyl-3-oxo-n-pent-1-yl, (4-hydroxynaph-2-yl)methyl, and the like.

The term “substituted naphthyl” means a naphthyl group substituted with one or more of the above-identified substituents, and the term “(1 or 2 naphthyl)alkyl” means a naphthyl (1 or 2) attached to one of the above-described alkyl groups.

The terms “halo” and “halogen” refer to the fluoro, chloro, bromo or iodo groups. These terms may also be used to describe one or more halogens, which are the same or different. Preferred halogens in the context of this invention are chloro and fluoro.

The term “aryl” refers to aromatic five and six membered carbocyclic rings. Six-membered rings are preferred.

The term “heteroaryl” refers to optionally substituted aromatic five-membered or six-membered heterocyclic rings that have 1 to 4 heteroatoms, such as oxygen, sulfur and/or nitrogen atoms, in particular nitrogen, either alone or in conjunction with sulfur or oxygen ring atoms.

The following ring systems are representative examples of the heterocyclic radicals denoted by the term “heteroaryl” (whether substituted or unsubstituted): thienyl, furyl, pyrrolyl, pyrrolidinyl, imidazolyl, isoxazolyl, triazolyl, thiadiazolyl, oxadiazolyl, tetrazolyl, thiatriazolyl, oxatriazolyl, pyridyl, pyrimidyl, pyrazinyl, pyridazinyl, oxazinyl, triazinyl, thiadiazinyl, tetrazolo, 1,5-[b]pyridazinyl and purinyl, as well as benzo-fused derivatives, for example, benzoxazolyl, benzothiazolyl, benzimidazolyl and indolyl.

Substituents for the above optionally substituted heteroaryl rings are from one to three halo, trihalomethyl, amino, protected amino, amino salts, mono-substituted amino, di-substituted amino, carboxy, protected carboxy, carboxylate salts, hydroxy, protected hydroxy, salts of a hydroxy group, lower alkoxy, lower alkylthio, lower alkyl, substituted lower alkyl, cycloalkyl, substituted cycloalkyl, (cycloalkyl)alkyl, substituted (cycloalkyl)alkyl, phenyl, substituted phenyl, phenylalkyl, and substituted phenylalkyl groups.

Substituents for the heteroaryl group are as defined above, or as set forth below. As used in conjunction with the above substituents for heteroaryl rings, “trihalomethyl” can be trifluoromethyl, trichloromethyl, tribromomethyl or triiodomethyl; “lower alkoxy means a C₁ to C₄ alkoxy group, similarly, “lower alkylthio” means a C₁ to C₄ alkylthio group. The term “substituted lower alkyl” means the above-defined lower alkyl group substituted from one to three times by a hydroxy, protected hydroxy, amino, protected amino, cyano, halo, trifluoromethyl, mono-substituted amino, di-substituted amino, lower alkoxy, lower alkylthio, carboxy, protected carboxy, or a carboxy, amino, and/or hydroxy salt.

As used in conjunction with the substituents for the heteroaryl rings, the terms “substituted (cycloalkyl)alkyl” and “substituted cycloalkyl” are as defined above substituted with the same groups as listed for a “substituted alkyl” group. The term “(monosubstituted)amino” refers to an amino group with one substituent chosen from the group consisting of phenyl, substituted phenyl, alkyl, substituted alkyl, C₁ to C₇ acyl, C₂ to C₇ alkenyl, C₂ to C₇ substituted alkenyl, C₂ to C₇ alkynyl, C₇ to C₁₆ alkylaryl, C₇ to C₁₆ substituted alkylaryl and heteroaryl group. The (monosubstituted)amino can additionally have an amino-protecting group as encompassed by the term “protected (monosubstituted)amino”. The term “(disubstituted)amino” refers to amino groups with two substituents chosen from the group consisting of phenyl, substituted phenyl, alkyl, substituted alkyl, C₁ to C₇ acyl, C₂ to C₇ alkenyl, C₂ to C₇ alkynyl, C₇ to C₁₆ substituted alkylaryl and heteroaryl. The two substituents can be the same or different. The term “heteroaryl(alkyl)” denotes an alkyl group as defined above, substituted at any position by a heteroaryl group, as above defined.

Furthermore, the above optionally substituted five-membered or six-membered heterocyclic rings, and the above cycloalkyl rings, can optionally be fused to an aromatic five-membered or six-membered ring system such as a pyridine or a triazole system, and preferably to a benzene ring.

The term “carboxy-protecting group” as used herein refers to one of the ester derivatives of the carboxylic acid group commonly employed to block or protect the carboxylic acid group while reactions are carried out on other functional groups on the compound. Examples of such carboxylic acid protecting groups include t-butyl, 4-nitrobenzyl, 4-methoxybenzyl, 3,4-dimethoxybenzyl, 2,4-dimethoxybenzyl, 2,4,6-trimethoxybenzyl, 2,4,6-trimethylbenzyl, pentamethylbenzyl, 3,4-methylenedioxybenzyl, benzhydryl, 4,4′dimethoxytrityl, 4,4′,4″-trimethoxytrityl, 2-phenylpropyl, trimethylsilyl, t-butyldimethylsilyl, phenacyl, 2,2,2-trichloroethyl, β-(trimethylsilyl)ethyl, β-(di(n-butyl)methylsilyl)ethyl, p-toluenesulfonylethyl, 4-nitrobenzylsulfonylethyl, allyl, cinnamyl, 1-(trimethylsilylmethyl)-propenyl and like moieties. The species of carboxy-protecting group employed is not critical so long as the derivatized carboxylic acid is stable to the conditions of subsequent reaction(s) and can be removed at the appropriate point without disrupting the remainer of the molecule.

The term “hydroxy-protecting group” refers to readily cleavable groups bonded to hydroxyl groups, such as the tetrahydropyranyl, 2-methoxyprop-2-yl, 1-ethoxyeth-1-yl, methoxymethyl, β-methoxyethoxymethyl, methylthiomethyl, t-butyl, t-amyl, trityl, 4-methoxytrityl, 4,4′-dimethoxytrityl, 4,4′,4″-trimethoxytrityl, benzyl, allyl, trimethylsilyl, (t-butyl)dimethylsilyl, 2,2,2-trichloroethoxycarbonyl, and the like.

The term “amino-protecting group” as used herein refers to substituents of the amino group commonly employed to block or protect the amino functionality while reacting other functional groups of the molecule. The term “protected (monosubstituted)amino” means there is an amino-protecting group on the monosubstituted amino nitrogen atom. Examples for such amino protecting groups are listed in WO 00/01666.

The terms “natural and unnatural amino acid” refer to both the naturally occurring amino acids and other non-proteinogenic α-amino acids commonly utilized by those in the peptide chemistry arts when preparing synthetic analogues of naturally occurring peptides, including D and L forms. The naturally occurring amino acids are glycine, alanine, valine, leucine, isoleucine, serine, methionine, theonine, phenylalanine, tyrosine, tryptophan, cysteine, proline, histidine, aspartic acid, asparagine, glutamic acid, glutamine, γ-carboxyglutamic acid, arginine, ornithine and lysine. Examples of unnatural α-amino acids include hydroxylysine, citrulline, kynurenine, (4-aminophenyl)alanine, 3-(2′-naphthyl)alanine, 3-(1′-naphthyl)alanine, methionine sulfone, (t-butyl)alanine, (t-butyl)glycine, 4-hydroxyphenyl-glycine, aminoalanine, phenylglycine, vinylalanine, propargyl-glycine, 1,2,4-triazolo-3-alanine, thyronine, 6-hydroxytryptophan, 5-hydroxytryptophan, 3-hydroxykynurenine, 3-aminotyrosine, trifluoromethylalanine, 2-thienylalanine, (2-(4-pyridyl)ethyl)cysteine, 3,4-dimethoxy-phenylalanine, 3-(2′-thiazolyl)alanine, ibotenic acid, 1-amino-1-cyclopentane-carboxylic acid, 1-amino-1-cyclohexanecarboxylic acid, quisqualic acid, 3-(trifluoromethylphenyl)alanine, (cyclohexyl)glycine, thiohistidine, 3-methoxytyrosine, norleucine, norvaline, alloisoleucine, homoarginine, thioproline, dehydroproline, hydroxyproline, homoproline, indoline-2-carboxylic acid, 1,2,3,4-tetrahydroisoquinoline-3-carboxylic acid, 1,2,3,4-tetrahydroquinoline-2-carboxylic acid, a-amino-n-butyric acid, cyclohexylalanine, 2-amino-3-phenylbutyric acid, phenylalanine substituted at the ortho, meta, or para position of the phenyl moiety with one or two of the following groups: a (C₁ to C₄ alkyl, a (C₁ to C₄)alkoxy, a halogen or a nitro group, or substituted once with a methylenedioxy group; b-2- and 3-thienylalanine; β-2- and 3-furanylalanine; β-2-, 3- and 4-pyridylalanine, β-(benzothienyl-2- and 3-yl)alanine; β-(1- and 2-naphthyl)alanine; O-alkylated derivatives of serine, threonine or tyrosine; S-alkylated cysteine, S-alkylated homocysteine, the O-sulfate, O-phosphate, and O-carboxylate esters of tyrosine, 3-(sulfo)tyrosine, 3-(carboxy)tyrosine, 3-(phospho)tyrosine, the 4-methane-sulfonic acid ester of tyrosine, 4-methanephosphonic acid ester of tyrosine, 3,5-diiodotyrosine, 3-nitrotyrosine, ε-alkyllysine, and δ-alkyl ornithine. Any of these α-amino acids may be substituted with a methyl group at the alpha position, a halogen at any position of the aromatic residue on the α-amino side chain, or an appropriate protective group at the O, N, or S atoms of the side chain residues. Appropriate protecting groups are discussed above.

Depending on the choice of solvent and other conditions known to the skilled person, these compounds may also take the ketal or acetal form, and the use of these forms in the combination of the invention is included in the invention.

These compounds can be prepared as described in WO 00/01666 or in U.S. Pat. No. 6,544,951, hereby incorporated by reference in their entirety.

Preferred subgroups are those listed in U.S. Pat. No. 6,544,951.

A preferred compound of formula I is selected from:
(3S)-3-[N—(N′-(2-benzylphenyl)Oxamyl)Valinyl]Amino-5-(2′,3′,5′,6′-Tetrafluorophenoxy)-4-Oxopentanoic acid;
(3S)-3-[N—(N′-(1-Naphthyl)Oxamyl)Valinyl]Amino-5-(2′,3′,5′,6′-Tetrafluorophenoxy)-4-Oxopentanoic acid;
(3S)-3-[N—(N′-(Benzyl)Oxamyl)Valinyl]Amino-5-(2′,3′,5′,6′-Tetrafluorophenoxy)-4-Oxopentanoic acid;
(3S)-3-[N—(N′-(2-phenoxyphenyl)Oxamyl)Valinyl]Amino-5-(2′,3′,5′,6′-Tetrafluorophenoxy)-4-Oxopentanoic acid;
(3S)-3-[N—(N′-(1-Naphthylmethyl)Oxamyl)Valinyl]Amino-5-(2′,3′,5′,6′-Tetrafluorophenoxy)-4-Oxopentanoic acid;
(3S)-3-[N—(N′-(4-Chloro-1-Naphthyl)Oxamyl)Valinyl]Amino-5-(2′,3′,5′,6′-Tetrafluorophenoxy)-4-Oxopentanoic acid;
(3S)-3-[N—(N′-(2-Anthryl)Oxamyl)Valinyl]Amino-5-(2′,3′,5′,6′-Tetrafluorophenoxy)-4-Oxopentanoic acid;
(3S)-3-[N—(N′-(Phenethyl)Oxamyl)Valinyl]Amino-5-(2′,3′,5′,6′-Tetrafluorophenoxy)-4-Oxopentanoic acid;
(3S)-3-[N—(N′-(Phenyl)Oxamyl)Valinyl]Amino-5-(2′,3′,5′,6′-Tetrafluorophenoxy)-4-Oxopentanoic acid;
(3S)-3-[N—(N′-(2-Phenylphenyl)Oxamyl)Valinyl]Amino-5-(2′,3′,5′,6′-Tetrafluorophenoxy)-4-Oxopentanoic acid;
(3S)-3-[N—(N′-(4-n-Heptylphenyl)Oxamyl)Valinyl]Amino-5-(2′,3′,5′,6′-Tetrafluorophenoxy)-4-Oxopentanoic acid;
(3S)-3-[N—(N′-(5,6,7,8-Tetrahydro-1-Naphthyl)Oxamyl)Valinyl]Amino-5-(2′,3′,5′,6′-Tetrafluorophenoxy)-4-Oxopentanoic acid;
(3S)-3-[N—(N′-(1-Adamantyl)Oxamyl)Valinyl]Amino-5-(2′,3′,5′,6′-Tetrafluorophenoxy)-4-Oxopentanoic acid;
(3S)-3-[N—(N′-(4-Fluorophenyl)Oxamyl)Valinyl]Amino-5-(2′,3′,5′,6′-Tetrafluorophenoxy)-4-Oxopentanoic acid;
(3S)-3-[N—(N′-(2-Naphthyl)Oxamyl)Valinyl]Amino-5-(2′,3′,5′,6′-Tetrafluorophenoxy)-4-Oxopentanoic acid;
(3S)-3-[N—(N′-(2-methoxyphenyl)Oxamyl)Valinyl]Amino-5-(2′,3′,5′,6′-Tetrafluorophenoxy)-4-Oxopentanoic acid;
(3S)-3-[N—(N′-(N″-N′″-Diphenylamoni)Oxamyl)Valinyl]Amino-5-(2′,3′,5′,6′-Tetrafluorophenoxy)-4-Oxopentanoic acid;
(3S)-3-[N—(N′-(4-Pyridinyl)Oxamyl)Valinyl]Amino-5-(2′,3′,5′,6′-Tetrafluorophenoxy)-4-Oxopentanoic acid;
(3S)-3-[N—(N′-(2-Pyrazinyl)Oxamyl)Valinyl]Amino-5-(2′,3′,5′,6′-Tetrafluorophenoxy)-4-Oxopentanoic acid;
(3S)-3-[N—(N′-(1,2,3,4-Tetrahydro-1-Naphthyl)Oxamyl)Valinyl]Amino-5-(2′,3′,5′,6′-Tetrafluorophenoxy)-4-Oxopentanoic acid;
(3S)-3-[N—(N′-(3,4,5-Trimethoxybenzyl)Oxamyl)Valinyl]Amino-5-(2′,3′,5′,6′-Tetrafluorophenoxy)-4-Oxopentanoic acid;
(3S)-3-[N—(N′-(Benzhydryl)Oxamyl)Valinyl]Amino-5-(2′,3′,5′,6′-Tetrafluorophenoxy)-4-Oxopentanoic acid;
(3S)-3-[N—(N′-(3-Phenoxylphenyl)Oxamyl)Valinyl]Amino-5-(2′,3′,5′,6′-Tetrafluorophenoxy)-4-Oxopentanoic acid;
(3S)-3-[N—(N′-(2-tert-Butylphenyl)Oxamyl)Valinyl]Amino-5-(2′,3′,5′,6′-Tetrafluorophenoxy)-4-Oxopentanoic acid;
(3S)-3-[N—(N′-(2-Pyridinyl)Oxamyl)Valinyl]Amino-5-(2′,3′,5′,6′-Tetrafluorophenoxy)-4-Oxopentanoic acid;
(3S)-3-[N—(N′-(2,3,5,6-Tetrafluoro-4-Pyridinyl)Oxamyl)Valinyl]Amino-5-(2′,3′,5′,6′-Tetrafluorophenoxy)-4-Oxopentanoic acid;
(3S)-3-[N—(N′-(2-iodophenyl)Oxamyl)Valinyl]Amino-5-(2′,3′,5′,6′-Tetrafluorophenoxy)-4-Oxopentanoic acid;
(3S)-3-[N—(N′-(2,6-Difluorophenyl)Oxamyl)Valinyl]Amino-5-(2′,3′,5′,6′-Tetrafluorophenoxy)-4-Oxopentanoic acid;
(3S)-3-[N—(N′-(2,5-Di-tert-Butylphenyl)Oxamyl)Valinyl]Amino-5-(2′,3′,5′,6′-Tetrafluorophenoxy)-4-Oxopentanoic acid;
(3S)-3-[N—(N′-(5-Indanyl)Oxamyl)Valinyl]Amino-5-(2′,3′,5′,6′-Tetrafluorophenoxy)-4-Oxopentanoic acid;
(3S)-3-[N—(N′-(Methyl)Oxamyl)Valinyl]Amino-5-(2′,3′,5′,6′-Tetrafluorophenoxy)-4-Oxopentanoic acid;
(3S)-3-[N—(N′-(n-Heptyl)Oxamyl)Valinyl]Amino-5-(2′,3′,5′,6′-Tetrafluorophenoxy)-4-Oxopentanoic acid;
(3S)-3-[N—(N′-(tert-Octyl)Oxamyl)Valinyl]Amino-5-(2′,3′,5′,6′-Tetrafluorophenoxy)-4-Oxopentanoic acid;
(3S)-3-[N—(N′-(Cyclohexyl)Oxamyl)Valinyl]Amino-5-(2′,3′,5′,6′-Tetrafluorophenoxy)-4-Oxopentanoic acid;
(3S)-3-[N—(N′-(5-Phenyl-3-Pyrazolyl)Oxamyl)Valinyl]Amino-5-(2′,3′,5′,6′-Tetrafluorophenoxy)-4-Oxopentanoic acid;
(3S)-3-[N—(N′-(2,3,4,5-Tetrafluorophenyl)Oxamyl)Valinyl]Amino-5-(2′,3′,5′,6′-Tetrafluorophenoxy)-4-Oxopentanoic acid;
(3S)-3-[N—(N′-(2,3,4,6-Tetrafluorophenyl)Oxamyl)Valinyl]Amino-5-(2′,3′,5′,6′-Tetrafluorophenoxy)-4-Oxopentanoic acid;
(3S)-3-[N—(N′-(2,3,5,6-Tetrachlorophenyl)Oxamyl)Valinyl]Amino-5-(2′,3′,5′,6′-Tetrafluorophenoxy)-4-Oxopentanoic acid;
(3S)-3-[N—(N′-(2,3,4,5,6-Pentafluorophenyl)Oxamyl)Valinyl]Amino-5-(2′,3′,5′,6′-Tetrafluorophenoxy)-4-Oxopentanoic acid;
(3S)-3-[N—(N′-(2-Benzimidazolyl)Oxamyl)Valinyl]Amino-5-(2′,3′,5′,6′-Tetrafluorophenoxy)-4-Oxopentanoic acid;
(3S)-3-[N—(N′-(1-Naphthyl)Oxamyl)Valinyl]Amino-5-(2′,6′-Difluorophenoxy)-4-Oxopentanoic acid;
(3S)-3-[N—(N′-(1-Naphthyl)Oxamyl)Valinyl]Amino-5-(2′,4′,6′-Trifluorophenoxy)-4-Oxopentanoic acid;
(3S)-3-[N—(N′-(1-Naphthyl)Oxamyl)Valinyl]Amino-5-(Diphenylphosphinyloxy)-4-Oxopentanoic acid;
(3S)-3-[N—(N′-(1-Naphthyl)Oxamyl)Valinyl]Amino-5-(Methylphenylphosphinyloxy)-4-Oxopentanoic acid;
(3RS)-3-[N—(N′-(1-Naphthyl)Oxamyl)Valinyl]Amino-5-Fluoro-4-Oxopentanoic acid;
(3S)-3-[N—(N′-(Phenyl)Oxamyl)Alaninyl]Amino-5-(2′,3′,5′,6′-Tetrafluorophenoxy)-4-Oxopentanoic acid;
(3S)-3-[N—(N′-(5,6,7,8-Tetrahydro-1-Naphthyl)Oxamyl)Alaninyl]Amino-5-(2′,3′,5′,6′-Tetrafluorophenoxy)-4-Oxopentanoic acid;
(3S)-3-[N—(N′-(2-Phenylphenyl)Oxamyl)Alaninyl]Amino-5-(2′,3′,5′,6′-Tetrafluorophenoxy)-4-Oxopentanoic acid;
(3S)-3-[N—(N′-(2-Benzylphenyl)Oxamyl)Alaninyl]Amino-5-(2′,3′,5′,6′-Tetrafluorophenoxy)-4-Oxopentanoic acid;
(3S)-3-[N—(N′-(1-Naphthyl methyl)Oxamyl)Alaninyl]Amino-5-(2′,3′,5′,6′-Tetrafluorophenoxy)-4-Oxopentanoic acid;
(3S)-3-[N—(N′-(2-Phenoxyphenyl)Oxamyl)Alaninyl]Amino-5-(2′,3′,5′,6′-Tetrafluorophenoxy)-4-Oxopentanoic acid;
(3S)-3-[N—(N′-(3-Phenoxylphenyl)Oxamyl)Alaninyl]Amino-5-(2′,3′,5′,6′-Tetrafluorophenoxy)-4-Oxopentanoic acid;
(3S)-3-[N—(N′-(4-Phenylphenyl)Oxamyl)Alaninyl]Amino-5-(2′,3′,5′,6′-Tetrafluorophenoxy)-4-Oxopentanoic acid;
(3S)-3-[N—(N′-(Benzhydryl)Oxamyl)Alaninyl]Amino-5-(2′,3′,5′,6′-Tetrafluorophenoxy)-4-Oxopentanoic acid;
(3S)-3-[N—(N′-(2-Phenylphenyl)Oxamyl)Alaninyl]Amino-5-(2′-Fluorophenoxy)-4-Oxopentanoic acid;
(3S)-3-[N—(N′-(2-tert-Butylphenyl)Oxamyl)Alaninyl]Amino-5-(2′-Fluorophenoxy)-4-Oxopentanoic acid;
(3S)-3-[N—(N′-(2-Phenylphenyl)Oxamyl)Alaninyl]Amino-5-(Diphenylphosphinoxy)-4-Oxopentanoic acid;
(3S)-3-[N—(N′-(1-Naphthyl)Oxamyl)Alaninyl]Amino-5-(Diphenylphosphinyloxy)-4-Oxopentanoic acid;
(3S)-3-[N—(N′-(1-Naphthyl)Oxamyl)Leucinyl]Amino-5-(2′,4′,6′-Trifluorophenoxy)-4-Oxopentanoic acid;
(3S)-3-[N—(N′-(1-Naphthyl)Oxamyl)(tert-Butyl)Glycinyl]Amino-5-(2′,3′,5′,6′-Tetrafluorophenoxy)-4-Oxopentanoic acid;
(3S)-3-[N—(N′-(1-Naphthyl)Oxamyl)(tert-Butyl)Norleucinyl]Amino-5-(2′,3′,5′,6′-Tetrafluorophenoxy)-4-Oxopentanoic acid;
(3S)-3-[N—(N′-(1-Naphthyl)Oxamyl)(tert-Butyl)Alaninyl]Amino-5-(2′,3′,5′,6′-Tetrafluorophenoxy)-4-Oxopentanoic acid;
(3S)-3-[N—(N′-(1-Naphthyl)Oxamyl)Leucinyl]Amino-5-(2′,3′,5′,6′-Tetrafluorophenoxy)-4-Oxopentanoic acid;
(3S)-3-[N—(N′-(1-Naphthyl)Oxamyl)Leucinyl]Amino-5-(Methylphenyl phosphinyloxy)-4-Oxopentanoic acid;
(3S)-3-[N—(N′-(1-Naphthyl)Oxamyl)Leucinyl]Amino-5-Fluoro-4-Oxopentanoic acid;
(3S)-3-[N—(N′-(1-Naphthyl)Oxamyl)Leucinyl]Amino-5-(Diphenylphosphinyloxy)-4-Oxopentanoic acid;
(3S)-3-[N—(N′-(2-(1H-Tetrazol-5-yl)Phenyl)Oxamyl)Valinyl]Amino-4-Oxobutanoic acid;
(3S)-3-[N—(N′-(1-Adamantyl)Phenyl)Oxamyl)Valinyl]Amino-4-Oxobutanoic acid;
(3S)-3-[N—(N′-(Phenyl)Oxamyl)Valinyl]Amino-4-Oxobutanoic acid;
(3S)-3-[N—(N′-(Benzyl)Oxamyl)Valinyl]Amino-4-Oxobutanoic acid;
(3S)-3-[N—(N′-(Phenethyl)Oxamyl)Valinyl]Amino-4-Oxobutanoic acid;
(3S)-3-[N—(N′-(2-Phenoxyphenyl)Oxamyl)Valinyl]Amino-4-Oxobutanoic acid;
(3S)-3-[N—(N′-(1-Naphthyl)Oxamyl)Valinyl]Amino-4-Oxobutanoic acid;
(3S)-3-[N—(N′-(2-Naphthyl)Oxamyl)Valinyl]Amino-4-Oxobutanoic acid;
(3S)-3-[N—(N′-(4-Chloro-1-Naphthyl)Oxamyl)Valinyl]Amino-4-Oxobutanoic acid;
(3S)-3-[N—(N′-(5,6,7,8-Tetrahydro-1-Naphthyl)Oxamyl)Valinyl]Amino-4-Oxobutanoic acid;
(3S)-3-[N—(N′-(1,2,3,4-Tetrahydro-1-Naphthyl)Oxamyl)Valinyl]Amino-4-Oxobutanoic acid;
(3S)-3-[N—(N′-(1-Naphthylmethyl)Oxamyl)Valinyl]Amino-4-Oxobutanoic acid;
(3S)-3-[N—(N′-(1-Naphthyl)Oxamyl)Leucinyl]Amino-4-Oxobutanoic acid;

Especially preferred are compounds selected from:
(3S)-3-[N—(N′-(2-Fluoro-4-Iodophenyl)Oxamyl)Valinyl]Amino-5-(2′,3′,5′,6′-Tetrafluorophenoxy)-4-Oxopentanoic acid;
(3S)-3-[N—(N′-(2-Chlorophenyl)Oxamyl)Valinyl]Amino-5-(2′,3′,5′,6′-Tetrafluorophenoxy)-4-Oxopentanoic acid;
(3S)-3-[N—(N′-(2-Bromophenyl)Oxamyl)Valinyl]Amino-5-(2′,3′, 5′,6′-Tetrafluorophenoxy)-4-Oxopentanoic acid;
(3S)-3-[N—(N′-(2-Fluorophenyl)Oxamyl)Valinyl]Amino-5-(2′,3′,5′,6′-Tetrafluorophenoxy)-4-Oxopentanoic acid;
(3S)-3-[N—(N′-(2-Trifluoromethylphenyl)Oxamyl)Valinyl]Amino-5-(2′,3′,5′,6′-Tetrafluorophenoxy)-4-Oxopentanoic acid;
(3S)-3-[N—(N′-(1-Anthryl)Oxamyl)Valinyl]Amino-5-(2′,3′,5′,6′-Tetrafluorophenoxy)-4-Oxopentanoic acid;
(3S)-3-[N—(N′-(2-Tert-Butylphenyl)Oxamyl)Alaninyl]Amino-5-(2′,3′,5′,6′-Tetrafluorophenoxy)-4-Oxopentanoic acid;
(3S)-3-[N—(N′-(2-Trifluoromethylphenyl)Oxamyl)Alaninyl]Amino-5-(2′,3′,5′,6′-Tetrafluorophenoxy)-4-Oxopentanoic acid;
(3S)-3-[N—(N′-(2,6-Difluorophenyl)Oxamyl)Alaninyl]Amino-5-(2′,3′,5′,6′-Tetrafluorophenoxy)-4-Oxopentanoic acid;
(3S)-3-[N—(N′-(1-Naphthyl)Oxamyl)Alaninyl]Amino-5-(2′,3′,5′,6′-Tetrafluorophenoxy)-4-Oxopentanoic acid;
(3S)-3-[N—(N′-(4-Methoxyphenyl)Oxamyl)Alaninyl]Amino-5-(2′,3′,5′,6′-Tetrafluorophenoxy)-4-Oxopentanoic acid;
(3S)-3-[N—(N′-(2-Trifluoromethylphenyl)Oxamyl)Valinyl]Amino-4-Oxobutanoic acid;
(3S)-3-[N—(N′-(2-tert-Butylmethylphenyl)Oxamyl)Valinyl]Amino-4-Oxobutanoic acid;
(3S)-3-[N—(N′-(2-Benzylphenyl)Oxamyl)Valinyl]Amino-4-Oxobutanoic acid;
(3S)-3-[N—(N′-(2-Phenylphenyl)Oxamyl)Valinyl]Amino-4-Oxobutanoic acid.

Most preferably, the compound is (3S)-3-[N—(N′-(2-Tert-Butylphenyl)Oxamyl)Alaninyl]Amino-5-(2′,3′,5′,6′-Tetrafluorophenoxy)-4-Oxopentanoic acid.

The anti-MAdCAM antibody or antigen binding portion thereof is preferably an antibody as disclosed in WO2005/067620, hereby incorporated by reference in its entirety. In particular, all SEQ ID Nos referred to herein relate to the sequences actually disclosed in WO2005/067620.

Preferably the anti-MAdCAM antibody used in the invention specifically binds MAdCAM. Even more preferably, at least the CDR sequences of said antibody are human CDR sequences, or an antigen-binding portion of a human antibody. Preferably the antibody is a human antibody, more preferably an antibody that acts as a MAdCAM antagonist.

Another aspect of the invention is the use in the combination of the invention of the heavy and/or light chain of said anti-MAdCAM antibody or the variable region or other antigen-binding portion thereof, or nucleic acid molecules encoding any of the foregoing and a pharmaceutically acceptable carrier. This aspect of the invention includes the use of fragments of any of the foregoing antibodies, including but not limited to Fab fragments, F(ab′)₂ fragments, single-chain Fv (scFv) fragments.

Preferably, the anti-MAdCAM antibody is a human inhibitory anti-MAdCAM antibody selected from 1.7.2, 1.8.2, 6.14.2, 6.22.2, 6.34.2, 6.67.1, 6.73.2, 6.77.1, 7.16.6, 7.20.5, 7.26.4, 9.8.2, 6.22.2-mod, 6.34.2-mod, 6.67.1-mod, 6.77.1-mod or 7.26.4-mod as disclosed in WO2005/067620. Preferably, the anti-MAdCAM antibody comprises a light chain comprising an amino acid sequence selected from SEQ ID NO: 4, 8, 12, 16, 20, 24, 28, 32, 36, 40, 44, 48, 54, 58, 62, 66 or 68 as disclosed in WO2005/067620 (with or without the signal sequence) or the variable region of any one of said amino acid sequences, or one or more CDRs from these amino acid sequences. The anti-MAdCAM antibody preferably comprises a heavy chain comprising an amino acid sequence selected from SEQ ID NO: 2, 6, 10, 14, 18, 22, 26, 30, 34, 38, 42, 46, 52, 56, 60 or 64 as disclosed in WO2005/067620 (with or without the signal sequence) or the amino acid sequence of the variable region, or of one or more CDRs from said amino acid sequences. The anti-MAdCAM antibody preferably is a human anti-MAdCAM antibody comprising the amino acid sequence from the beginning of the CDR1 to the end of the CDR3 of any one of the above-mentioned sequences. The anti-MAdCAM antibody used in the invention can also be an anti-MAdCAM antibody comprising one or more FR regions of any of the above-mentioned sequences.

The anti-MAdCAM antibody used in the combination of the invention can also include an anti-MAdCAM antibody comprising one of the afore-mentioned amino acid sequences in which one or more modifications have been made. For example, cysteines in the antibody, which may be chemically reactive, are substituted with another residue, such as, without limitation, alanine or serine. The substitution can be at a non-canonical cysteine or at a canonical cysteine. The substitution can be made in a CDR or framework region of a variable domain or in the constant domain of an antibody.

An amino acid substitution may also be made to eliminate potential proteolytic sites in the antibody. Such sites may occur in a CDR or framework region of a variable domain or in the constant domain of an antibody. Substitution of cysteine residues and removal of proteolytic sites may decrease the heterogeneity in the antibody product. Asparagine-glycine pairs, which form potential deamidation sites, may be eliminated by altering one or both of the residues. An amino acid substitution may be made to add or to remove potential glycosylation sites in the variable region of an antibody used in the invention.

The C-terminal lysine of the heavy chain of the anti-MAdCAM antibody used the invention may be cleaved. The heavy and light chains of the anti-MAdCAM antibodies may optionally include a signal sequence.

Twelve preferred inhibitory human anti-MAdCAM monoclonal antibodies for use in the combination of the invention are described in detail in WO2005/067620: 1.7.2, 1.8.2, 6.14.2, 6.22.2, 6.34.2, 6.67.1, 6.73.2, 6.77.1, 7.16.6, 7.20.5, 7.26.4 and 9.8.2.

### Class and Subclass of Anti-MAdCAM Antibodies

The antibody may be an IgG, an IgM, an IgE, an IgA or an IgD molecule. Preferably the antibody is an IgG class and is an IgG₁, IgG₂, IgG₃ or IgG₄ subclass. More preferably, the anti-MAdCAM antibody is subclass IgG₂ or IgG₄. More preferably, the anti-MAdCAM antibody is the same class and subclass as antibody 1.7.2, 1.8.2, 7.16.6, 7.20.5, 7.26.4, 6.22.2-mod, 6.34.2-mod, 6.67.1-mod, 6.77.1-mod or 7.26.4-mod which is IgG₂, or 6.14.2, 6.22.2, 6.34.2, 6.67.1, 6.73.2, 6.77.1 or 9.8.2, which is IgG₄ as described in WO2005/067620.

The class and subclass of anti-MAdCAM antibodies may be determined by any method known in the art. In general, the class and subclass of an antibody may be determined using antibodies that are specific for a particular class and subclass of antibody. Such antibodies are available commercially. ELISA, Western Blot as well as other techniques can determine the class and subclass. Alternatively, the class and subclass may be determined by sequencing all or a portion of the constant domains of the heavy and/or light chains of the antibodies, comparing their amino acid sequences to the known amino acid sequences of various classes and subclasses of immunoglobulins, and determining the class and subclass of the antibodies as the class showing the highest sequence identity.

### Species and Molecule Selectivity

The anti-MAdCAM antibody used in the combination of the invention demonstrates both species and molecule selectivity. The anti-MAdCAM antibody may bind to human, cynomolgus or dog MAdCAM. Other anti-MAdCAM antibodies used in the combination of the invention do not bind to a New World monkey species such as a marmoset. One may determine the species selectivity for the anti-MAdCAM antibody using methods well known in the art. For instance, one may determine species selectivity using Western blot, FACS, ELISA or immunohistochemistry. In a preferred embodiment, one may determine the species selectivity using immunohistochemistry.

An anti-MAdCAM antibody used in the combination of the invention that specifically binds MAdCAM has selectivity for MAdCAM over VCAM, fibronectin or any other antigen that is at least 10 fold, preferably at least 20, 30, 40, 50, 60, 70, 80 or 90 fold, most preferably at least 100 fold. Preferably the anti-MAdCAM antibody does not exhibit any appreciable binding to VCAM, fibronectin or any other antigen other than MAdCAM. One may determine the selectivity of the anti-MAdCAM antibody for MAdCAM using methods well known in the art following the teachings of the specification. For instance, one may determine the selectivity using Western blot, FACS, ELISA, or immunohistochemistry.

### Binding Affinity of Anti-MAdCAM Antibodies to MAdCAM

The anti-MAdCAM antibodies used in the combination of the invention preferably specifically bind to MAdCAM with high affinity. One anti-MAdCAM antibody used in the combination of the invention specifically binds to MAdCAM with a K_{d} of 3×10⁻⁸ M or less, as measured by surface plasmon resonance, such as BIAcore. Preferably, the antibody specifically binds to MAdCAM with a K_{d} of 1×10⁻⁸ or less or 1×10⁻⁹ M or less. More preferably, the antibody specifically binds to MAdCAM with a K_{d} or 1×10⁻¹⁰ M or less. An antibody used in the combination specifically binds to MAdCAM with a K_{d} of 2.66×10⁻¹⁰M or less, 2.35×10⁻¹¹M or less or 9×10⁻¹²M or less. Preferably, the antibody specifically binds to MAdCAM with a K_{d} or 1×10⁻¹¹ M or less. Preferably, the antibody specifically binds to MAdCAM with substantially the same K_{d} as an antibody selected from 1.7.2, 1.8.2, 6.14.2, 6.22.2, 6.34.2, 6.67.1, 6.73.2, 6.77.1, 7.16.6, 7.20.5, 7.26.4, 9.8.2, 6.22.2-mod, 6.34.2-mod, 6.67.1-mod, 6.77.1-mod or 7.26.4-mod. An antibody with “substantially the same K_{d}” as a reference antibody has a K_{d} that is ±100 pM, preferably ±50 pM, more preferably ±20 pM, still more preferably ±10 pM, ±5 pM or ±2 pM, compared to the K_{d} of the reference antibody in the same experiment. Preferably, the antibody binds to MAdCAM with substantially the same K_{d} as an antibody that comprises one or more variable domains or one or more CDRs from an antibody selected from 1.7.2, 1.8.2, 6.14.2, 6.22.2, 6.34.2, 6.67.1, 6.73.2, 6.77.1, 7.16.6, 7.20.5, 7.26.4, 9.8.2, 6.22.2-mod, 6.34.2-mod, 6.67.1-mod, 6.77.1-mod or 7.26.4-mod as disclosed in WO2005/067620. Preferably, the antibody binds to MAdCAM with substantially the same K_{d} as an antibody that comprises one of the amino acid sequences selected from SEQ ID NOS: 2, 4, 6, 8, 10, 12, 14, 16, 18, 20, 22, 24, 26, 28, 30, 32, 34, 36, 38, 40, 42, 44, 46 48, 52, 54, 56, 58, 62, 64, 66 or 68 (with or without the signal sequence) as disclosed in WO2005/067620, or the variable domain thereof. Preferably, the antibody binds to MAdCAM with substantially the same K_{d} as an antibody that comprises one or more CDRs from an antibody that comprises an amino acid sequence selected from SEQ ID NOS: 2, 4, 6, 8, 10, 12, 14, 16, 18, 20, 22, 24, 26, 28, 30, 32, 34, 36, 38, 40, 42, 44, 46 48, 52, 54, 56, 58, 62, 64, 66 or 68 as disclosed in WO2005/067620.

The binding affinity of an anti-MAdCAM antibody to MAdCAM may be determined by any method known in the art. In one embodiment, the binding affinity can be measured by competitive ELISAs, RIAs or surface plasmon resonance, such as BIAcore. In a more preferred embodiment, the binding affinity is measured by surface plasmon resonance. In an even more preferred embodiment, the binding affinity and dissociation rate is measured using a BIAcore. An example of determining binding affinity can be found in WO2005/067620.

### Half-Life of Anti-MAdCAM Antibodies

The anti-MAdCAM antibody used in the combination of the invention has a half-life of at least one day in vitro or in vivo. Preferably, the antibody or portion thereof has a half-life of at least three days. More preferably, the antibody or portion thereof has a half-life of four days or longer. Even more preferably, the antibody or portion thereof has a half-life of eight days or longer. The antibody or antigen-binding portion thereof used in the invention may also be derivatized or modified such that it has a longer half-life, as discussed below. In another preferred embodiment, the antibody may contain point mutations to increase serum half life, such as described WO 00/09560.

The antibody half-life may be measured by any means known to one having ordinary skill in the art. For instance, the antibody half life may be measured by Western blot, ELISA or RIA over an appropriate period of time. The antibody half-life may be measured in any appropriate animal, such as a primate, e.g., cynomolgus monkey, or a human.

### Identification of MAdCAM Epitopes Recognized by Anti-MAdCAM Antibody

The invention also provides a combination with an anti-fibrotic agent of a human anti-MAdCAM antibody that binds the same antigen or epitope as a human anti-MAdCAM antibody provided herein. Further, the invention provides the combination with an anti-fibrotic agent of a human anti-MAdCAM antibody that competes or cross-competes with a human anti-MAdCAM antibody. Preferably, the human anti-MAdCAM antibody is 1.7.2, 1.8.2, 6.14.2, 6.22.2, 6.34.2, 6.67.1, 6.73.2, 6.77.1, 7.16.6, 7.20.5, 7.26.4, 9.8.2, 6.22.2-mod, 6.34.2-mod, 6.67.1-mod, 6.77.1-mod or 7.26.4-mod. Preferably, the human anti-MAdCAM antibody comprises one or more variable domains or one or more CDRs from an antibody selected from 1.7.2, 1.8.2, 6.14.2, 6.22.2, 6.34.2, 6.67.1, 6.73.2, 6.77.1, 7.16.6, 7.20.5, 7.26.4, 9.8.2, 6.22.2-mod, 6.34.2-mod, 6.67.1-mod, 6.77.1-mod or 7.26.4-mod. Preferably, the human anti-MAdCAM antibody comprises one of the amino acid sequences selected from SEQ ID NOS: 2, 4, 6, 8, 10, 12, 14, 16, 18, 20, 22, 24, 26, 28, 30, 32, 34, 36, 38, 40, 42, 44, 46 48, 52, 54, 56, 58, 62, 64, 66 or 68 (with or without the signal sequence) as described in WO2005/067620, or a variable domain thereof. Preferably, the human anti-MAdCAM antibody comprises one or more CDRs from an antibody that comprises one of the amino acid sequences selected from SEQ ID NOS: 2, 4, 6, 8, 10, 12, 14, 16, 18, 20, 22, 24, 26, 28, 30, 32, 34, 36, 38, 40, 42, 44, 46 48, 52, 54, 56, 58, 62, 64, 66 or 68, as described in WO2005/067620.

One may determine whether an anti-MAdCAM antibody binds to the same antigen as another anti-MAdCAM antibody using a variety of methods known in the art. For instance, one can use a known anti-MAdCAM antibody to capture the antigen, elute the antigen from the anti-MAdCAM antibody, and then determine whether the test antibody will bind to the eluted antigen. One may determine whether an antibody competes with an anti-MAdCAM antibody by binding the anti-MAdCAM antibody to MAdCAM under saturating conditions, and then measuring the ability of the test antibody to bind to MAdCAM. If the test antibody is able to bind to the MAdCAM at the same time as the anti-MAdCAM antibody, then the test antibody binds to a different epitope than the anti-MAdCAM antibody. However, if the test antibody is not able to bind to the MAdCAM at the same time, then the test antibody competes with the human anti-MAdCAM antibody. This experiment may be performed using ELISA, or surface plasmon resonance or, preferably, BIAcore. To test whether an anti-MAdCAM antibody cross-competes with another anti-MAdCAM antibody, one may use the competition method described above in two directions, i.e. determining if the known antibody blocks the test antibody and vice versa.

### Light and Heavy Chain Gene Usage

The invention also provides the combination with an anti-fibrotic agent of an anti-MAdCAM antibody that comprises a light chain variable region encoded by a human κ gene. Preferably, the light chain variable region is encoded by a human Vκ A2, A3, A26, B3, O12 or O18 gene family. Preferably, the light chain comprises no more than eleven, no more than six or no more than three amino acid substitutions from the germline human Vκ A2, A3, A26, B3, O12 or O18 sequence. Preferably, the amino acid substitutions are conservative substitutions.

Preferably, the VL of the anti-MAdCAM antibody contains the same mutations, relative to the germline amino acid sequence, as any one or more of the VL of antibodies 1.7.2, 1.8.2, 6.14.2, 6.22.2, 6.34.2, 6.67.1, 6.73.2, 6.77.1, 7.16.6, 7.20.5, 7.26.4, 9.8.2, 6.22.2-mod, 6.34.2-mod, 6.67.1-mod, 6.77.1-mod or 7.26.4-mod. The invention includes the combination with an anti-fibrotic agent of an anti-MAdCAM antibody that utilizes the same human Vκ and human Jκ genes as an exemplified antibody. The antibody may comprise one or more of the same mutations from germline as one or more exemplified antibodies, or the antibody may comprise different substitutions at one or more of the same positions as one or more of the exemplified antibodies. For example, the VL of the anti-MAdCAM antibody may contain one or more amino acid substitutions that are the same as those present in antibody 7.16.6, and another amino acid substitution that is the same as antibody 7.26.4. In this manner, one can mix and match different features of antibody binding in order to alter, e.g., the affinity of the antibody for MAdCAM or its dissociation rate from the antigen. The mutations may be made in the same position as those found in any one or more of the VL of antibodies 1.7.2, 1.8.2, 6.14.2, 6.22.2, 6.34.2, 6.67.1, 6.73.2, 6.77.1, 7.16.6, 7.20.5, 7.26.4, 9.8.2, 6.22.2-mod, 6.34.2-mod, 6.67.1-mod, 6.77.1-mod or 7.26.4-mod, but conservative amino acid substitutions are made rather than using the same amino acid. For example, if the amino acid substitution compared to the germine in one of the antibodies 1.7.2, 1.8.2, 6.14.2, 6.22.2, 6.34.2, 6.67.1, 6.73.2, 6.77.1, 7.16.6, 7.20.5, 7.26.4, 9.8.2, 6.22.2-mod, 6.34.2-mod, 6.67.1-mod, 6.77.1-mod or 7.26.4-mod is glutamate, one may conservatively substitute aspartate. Similarly, if the amino acid substitution is serine, one may conservatively substitute threonine.

The light chain of the anti-MAdCAM antibody may comprise an amino acid sequence that is the same as the amino acid sequence of the VL of 1.7.2, 1.8.2, 6.14.2, 6.22.2, 6.34.2, 6.67.1, 6.73.2, 6.77.1, 7.16.6, 7.20.5, 7.26.4, 9.8.2, 6.22.2-mod, 6.34.2-mod, 6.67.1-mod, 6.77.1-mod or 7.26.4-mod. The light chain preferably comprises amino acid sequences that are the same as the CDR regions of the light chain of 1.7.2, 1.8.2, 6.14.2, 6.22.2, 6.34.2, 6.67.1, 6.73.2, 6.77.1, 7.16.6, 7.20.5, 7.26.4, 9.8.2, 6.22.2-mod, 6.34.2-mod, 6.67.1-mod, 6.77.1-mod or 7.26.4-mod. The light chain may comprise an amino acid sequence with at least one CDR region of the light chain of 1.7.2, 1.8.2, 6.14.2, 6.22.2, 6.34.2, 6.67.1, 6.73.2, 6.77.1, 7.16.6, 7.20.5, 7.26.4, 9.8.2, 6.22.2-mod, 6.34.2-mod, 6.67.1-mod, 6.77.1-mod or 7.26.4-mod. The light chain may comprise amino acid sequences with CDRs from different light chains that use the same Vκ and Jκ genes. Preferably the CDRs from different light chains are obtained from 1.7.2, 1.8.2, 6.14.2, 6.22.2, 6.34.2, 6.67.1, 6.73.2, 6.77.1, 7.16.6, 7.20.5, 7.26.4, 9.8.2, 6.22.2-mod, 6.34.2-mod, 6.67.1-mod, 6.77.1-mod or 7.26.4-mod. Preferably the light chain comprises an amino acid sequence selected from SEQ ID NOS: 4, 8, 12, 16, 20, 24, 28, 32, 36, 40, 44, 48, 54, 58, 62, 64, 66 or 68 as described in WO2005/067620 with or without the signal sequence. Preferably the light chain comprises an amino acid sequence encoded by a nucleotide sequence selected from SEQ ID NOS: 3, 7, 11, 15, 19, 23, 27, 31, 35, 39, 43, 47, 53, 57, 61, 65 or 67 (with or without the signal sequence) as described in WO2005/067620, or a nucleotide sequence that encodes an amino acid sequence having 1-11 amino acid insertions, deletions or substitutions therefrom. Preferably, the amino acid substitutions are conservative amino acid substitutions. The antibody or portion thereof may comprise a lambda light chain.

The present invention also provides the combination with an anti-fibrotic agent of an anti-MAdCAM antibody or portion thereof that comprises a human VH gene sequence or a sequence derived from a human VH gene. The heavy chain amino acid sequence may be derived from a human VH 1-18, 3-15, 3-21, 3-23, 3-30, 3-33 or 4-4 gene family. Preferably, the heavy chain comprises no more than fifteen, no more than six or no more than three amino acid changes from germine human VH 1-18, 3-15, 3-21, 3-23, 3-30, 3-33 or 4-4 gene sequence.

SEQ ID NOS: 2, 6, 10, 14, 18, 22, 26, 30, 34, 38, 42 and 46 as described in WO2005/067620 provide the amino acid sequences of the full-length heavy chains of twelve anti-MAdCAM antibodies that can be used in the combination of the invention.

Preferably, the VH of the anti-MAdCAM antibody contains the same mutations, relative to the germline amino acid sequence, as any one or more of the VH of antibodies 1.7.2, 1.8.2, 6.14.2, 6.22.2, 6.34.2, 6.67.1, 6.73.2, 6.77.1, 7.16.6, 7.20.5, 7.26.4, 9.8.2, 6.22.2-mod, 6.34.2-mod, 6.67.1-mod, 6.77.1-mod or 7.26.4-mod. Similar to that discussed above, the antibody comprises one or more of the same mutations from germline as one or more exemplified antibodies. The antibody may also comprise different substitutions at one or more of the same positions as one or more of the exemplified antibodies. For example, the VH of the anti-MAdCAM antibody may contain one or more amino acid substitutions that are the same as those present in antibody 7.16.6, and another amino acid substitution that is the same as antibody 7.26.4. In this manner, one can mix and match different features of antibody binding in order to alter, e.g., the affinity of the antibody for MAdCAM or its dissociation rate from the antigen. An amino acid substitution compared to germline may be made at the same position as a substitution from germine as found in any one or more of the VH of reference antibody 1.7.2, 1.8.2, 6.14.2, 6.22.2, 6.34.2, 6.67.1, 6.73.2, 6.77.1, 7.16.6, 7.20.5, 7.26.4, 9.8.2, 6.22.2-mod, 6.34.2-mod, 6.67.1-mod, 6.77.1-mod or 7.26.4-mod, but the position is substituted with a different residue, which is a conservative substitution compared to the reference antibody.

Preferably the heavy chain of the anti-MAdCAM antibody used in the combination of the invention comprises an amino acid sequence that is the same as the amino acid sequence of the VH of 1.7.2, 1.8.2, 6.14.2, 6.22.2, 6.34.2, 6.67.1, 6.73.2, 6.77.1, 7.16.6, 7.20.5, 7.26.4, 9.8.2, 6.22.2-mod, 6.34.2-mod, 6.67.1-mod, 6.77.1-mod or 7.26.4-mod. More preferably, the heavy chain comprises amino acid sequences that are the same as the CDR regions of the heavy chain of 1.7.2, 1.8.2, 6.14.2, 6.22.2, 6.34.2, 6.67.1, 6.73.2, 6.77.1, 7.16.6, 7.20.5, 7.26.4, 9.8.2, 6.22.2-mod, 6.34.2-mod, 6.67.1-mod, 6.77.1-mod or 7.26.4-mod. Preferably, the heavy chain comprises an amino acid sequence from at least one CDR region of the heavy chain of 1.7.2, 1.8.2, 6.14.2, 6.22.2, 6.34.2, 6.67.1, 6.73.2, 6.77.1, 7.16.6, 7.20.4, 7.26.4, 9.8.2, 6.22.2-mod, 6.34.2-mod, 6.67.1-mod, 6.77.1-mod or 7.26.4-mod, or the heavy chain may comprise amino acid sequences with CDRs from different heavy chains. Preferably, the CDRs from different heavy chains are obtained from 1.7.2, 1.8.2, 6.14.2, 6.22.2, 6.34.2, 6.67.1, 6.73.2, 6.77.1, 7.16.6, 7.20.5, 7.26.4, 9.8.2, 6.22.2-mod, 6.34.2-mod, 6.67.1-mod, 6.77.1-mod or 7.26.4-mod. Preferably, the heavy chain comprises an amino acid sequence selected from SEQ ID NOS: 2, 6, 10, 14, 18, 22, 26, 30, 34, 38, 42, 46, 52, 56, 60 or 64 as described in WO2005/067620 with or without the signal sequence. The heavy chain may also comprise an amino acid sequence encoded by a nucleotide sequence selected from SEQ ID NOS: 1, 5, 9, 13, 17, 21, 25, 29, 33, 37, 41, 45, 51, 55, 59 or 63 as described in WO2005/067620, or a nucleotide sequence that encodes an amino acid sequence having 1-15 amino acid insertions, deletions or substitutions therefrom. The substitutions are preferably conservative amino acid substitutions.

### Nucleic Acids, Vectors, Host Cells and Recombinant Methods of Making Antibodies

The nucleic acids, vectors, host cells and recombinant methods of making these antibodies are described in WO 2005/067620.

### Derivatized and Labeled Antibodies

An antibody or antibody portion for the combination of the invention can be derivatized or linked to another molecule (e.g., another peptide or protein). In general, the antibodies or portions thereof are derivatized such that the MAdCAM binding is not affected adversely by the derivatization or labeling. Accordingly, the antibodies and antibody portions for the combination of the invention are intended to include both intact and modified forms of the human anti-MAdCAM antibodies described herein. For example, an antibody or antibody portion used in the invention can be functionally linked (by chemical coupling, genetic fusion, noncovalent association or otherwise) to one or more other molecular entities, such as another antibody (e.g., a bispecific antibody or a diabody), a detection agent, a cytotoxic agent, a pharmaceutical agent, and/or a protein or peptide that can mediate association of the antibody or antibody portion with another molecule (such as a streptavidin core region or a polyhistidine tag).

One type of derivatized antibody is produced by crosslinking two or more antibodies (of the same type or of different types, e.g., to create bispecific antibodies). Suitable crosslinkers include those that are heterobifunctional, having two distinctly reactive groups separated by an appropriate spacer (e.g., m-maleimidobenzoyl-N-hydroxysuccinimide ester) or homobifunctional (e.g., disuccinimidyl suberate). Such linkers are available from Pierce Chemical Company, Rockford, Ill.

Another type of derivatized antibody is a labeled antibody. Useful detection agents with which an antibody or antibody portion of the invention may be derivatized include fluorescent compounds, including fluorescein, fluorescein isothiocyanate, rhodamine, 5-dimethylamine-1-napthalenesulfonyl chloride, phycoerythrin, lanthanide phosphors and the like. An antibody may also be labeled with enzymes that are useful for detection, such as horseradish peroxidase, β-galactosidase, luciferase, alkaline phosphatase, glucose oxidase and the like. When an antibody is labeled with a detectable enzyme, it is detected by adding additional reagents that the enzyme uses to produce a reaction product that can be discerned. For example, when the agent horseradish peroxidase is present, the addition of hydrogen peroxide and diaminobenzidine leads to a colored reaction product, which is detectable. An antibody may also be labeled with biotin, and detected through indirect measurement of avidin or streptavidin binding. An antibody may be labeled with a magnetic agent, such as gadolinium. An antibody may also be labeled with a predetermined polypeptide epitope recognized by a secondary reporter (e.g., leucine zipper pair sequences, binding sites for secondary antibodies, metal binding domains, epitope tags). In some embodiments, labels are attached by spacer arms of various lengths to reduce potential steric hindrance.

An anti-MAdCAM antibody may also be labeled with a radiolabeled amino acid. The radiolabel may be used for both diagnostic and therapeutic purposes. For instance, the radiolabel may be used to detect MAdCAM-expressing tissues by x-ray or other diagnostic techniques. Further, the radiolabel may be used therapeutically as a toxin for diseased tissue or MAdCAM expressing tumors. Examples of labels for polypeptides include, but are not limited to, the following radioisotopes or radionuclides—³H, ¹⁴C, ¹⁵N, ³⁵S, ⁹⁰Y, ⁹⁹Tc, ¹¹¹In, ¹²⁵I, ¹³¹I.

An anti-MAdCAM antibody may also be derivatized with a chemical group such as polyethylene glycol (PEG), a methyl or ethyl group, or a carbohydrate group. These groups may be useful to improve the biological characteristics of the antibody, e.g., to increase serum half-life or to increase tissue binding. This methodology would also apply to any antigen-binding fragments or versions of anti-MAdCAM antibodies.

### Pharmaceutical Compositions and Kits

In a further aspect, the invention provides compositions comprising an inhibitory human anti-MAdCAM antibody and methods for treating subjects with such compositions. In some embodiments, the subject of treatment is human. In other embodiments, the subject is a veterinary subject. In some embodiments, the veterinary subject is a dog or a non-human primate.

Treatment may involve administration of one or more inhibitory anti-MAdCAM monoclonal antibodies, or antigen-binding fragments thereof, alone or with a pharmaceutically acceptable carrier. Inhibitory anti-MAdCAM antibodies and compositions comprising them, can be administered in combination with one or more other therapeutic, diagnostic or prophylactic agents.

As used herein, “pharmaceutically acceptable carrier” means any and all solvents, dispersion media, coatings, antibacterial and antifungal agents, isotonic and absorption enhancing or delaying agents, and the like that are physiologically compatible. Some examples of pharmaceutically acceptable carriers are water, saline, phosphate buffered saline, acetate buffer with sodium chloride, dextrose, glycerol, Polyethylene glycol, ethanol and the like, as well as combinations thereof. In many cases, it will be preferable to include isotonic agents, for example, sugars, polyalcohols such as mannitol, sorbitol, or sodium chloride in the composition. Additional examples of pharmaceutically acceptable substances are surfectants, wetting agents or minor amounts of auxiliary substances such as wetting or emulsifying agents, preservatives or buffers, which enhance the shelf life or effectiveness of the antibody.

The compositions used in this invention may be in a variety of forms, for example, liquid, semi-solid and solid dosage forms, such as liquid solutions (e.g., injectable and infusible solutions), dispersions or suspensions, tablets, pills, lyophilized cake, dry powders, liposomes and suppositories. The preferred form depends on the intended mode of administration and therapeutic application. Typical preferred compositions are in the form of injectable or infusible solutions, such as compositions similar to those used for passive immunization of humans. The preferred mode of administration is parenteral (e.g., intravenous, subcutaneous, intraperitoneal, intramuscular, intradermal). In a preferred embodiment, the antibody is administered by intravenous infusion or injection. In another preferred embodiment, the antibody is administered by intramuscular, intradermal or subcutaneous injection. If desired, the antibody may be administered by using a pump, enema, suppository, or indwelling reservoir or such like.

Therapeutic compositions typically must be sterile and stable under the conditions of manufacture and storage. The composition can be formulated as a solution, lyophilized cake, dry powder, microemulsion, dispersion, liposome, or other ordered structure suitable to high drug concentration. Sterile injectable solutions can be prepared by incorporating the anti-MAdCAM antibody in the required amount in an appropriate solvent with one or a combination of ingredients enumerated above, as required, followed by sterilization. In the case of sterile powders for the preparation of sterile injectable solutions, the preferred methods of preparation are vacuum drying and freeze-drying that yields a powder of the active ingredient plus any additional desired ingredient from a previously sterile solution thereof. Generally, dispersions are prepared by incorporating the active compound into a sterile vehicle that contains a basic dispersion medium and the required other ingredients from those enumerated above. The desired characteristics of a solution can be maintained, for example, by the use of surfactants and the required particle size in the case of dispersion by the use of surfactants, phospholipids and polymers. Prolonged absorption of injectable compositions can be brought about by including in the composition an agent that delays absorption, for example, monostearate salts, polymeric materials, oils and gelatin.

The antibodies of the combination of the present invention can be administered by a variety of methods known in the art, although for many therapeutic applications, the preferred route/mode of administration is subcutaneous, intramuscular, intradermal or intravenous infusion. As will be appreciated by the skilled artisan, the route and/or mode of administration will vary depending upon the desired results.

In certain embodiments, the antibody compositions may be prepared with a carrier that will protect the antibody against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems (J. R. Robinson, ed., Marcel Dekker, Inc., New York (1978)).

In certain embodiments, an anti-MAdCAM antibody of the combination of the invention can be orally administered, for example, with an inert diluent or an assimilable edible carrier. The compound (and other ingredients, if desired) can also be enclosed in a hard or soft shell gelatin capsule, compressed into tablets, or incorporated directly into the subject's diet. For oral therapeutic administration, the anti-MAdCAM antibodies can be incorporated with excipients and used in the form of ingestible tablets, buccal tablets, troches, capsules, elixirs, suspensions, syrups, wafers, and the like. To administer a compound of the invention by other than parenteral administration, it may be necessary to coat the compound with, or co-administer the compound with, a material to prevent its inactivation.

The compositions of the invention may include a “therapeutically effective amount” or a “prophylactically effective amount” of an antibody or antigen-binding portion of the invention. A “therapeutically effective amount” refers to an amount effective, at dosages and for periods of time necessary, to achieve the desired therapeutic result. A therapeutically effective amount of the antibody or antibody portion may vary according to factors such as the disease state, age, sex, and weight of the individual, and the ability of the antibody or antibody portion to elicit a desired response in the individual. A therapeutically effective amount is also one in which any toxic or detrimental effects of the antibody or antibody portion are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount may be less than the therapeutically effective amount.

Dosage regimens can be adjusted to provide the optimum desired response (e.g., a therapeutic or prophylactic response). For example, a single bolus can be administered, several divided doses can be administered over time or the dose can be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a pre-determined quantity of active compound calculated to produce the desired therapeutic effect in association with the required pharmaceutical carrier. The specification for the dosage unit forms of the invention are dictated by and directly dependent on (a) the unique characteristics of the anti-MAdCAM antibody or portion thereof and the particular therapeutic or prophylactic effect to be achieved, and (b) the limitations inherent in the art of compounding such an antibody for the treatment of sensitivity in individuals.

An exemplary, non-limiting range for a therapeutically or prophylactically effective amount of an antibody or antibody portion of the invention is 0.025 to 50 mg/kg, more preferably 0.1 to 50 mg/kg, more preferably 0.1-25, 0.1 to 10 or 0.1 to 3 mg/kg. In some embodiments, a formulation contains 5 mg/mL of antibody in a buffer of 20 mM sodium acetate, pH 5.5, 140 mM NaCl, and 0.2 mg/mL polysorbate 80. In other embodiments, a formulation contains 10 mg/ml of antibody in 2.73 mg/ml of sodium acetate trihydrate, 45 mg/ml of mannitol, 0.02 mg/ml of disodium EDTA dihydrate, 0.2 mg/ml of polysorbate 80, adjusted to pH 5.5 with glacial acetic acid, e.g. for intravenous use. In other embodiments, a formulation contains 50 mg/ml of antibody, 2.73 mg/ml of sodium acetate trihydrate, 45 mg/ml of mannitol, 0.02 mg/ml of disodium EDTA dihydrate, 0.4 mg/ml of polysorbate 80, adjusted to pH 5.5 with glacial acetic acid, e.g. for subcutaneous or intradermal use. It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

Another aspect of the present invention provides kits comprising an anti-MAdCAM antibody or antibody portion of the invention or a composition comprising such an antibody with an anti-fibrotic agent as disclosed herein. A kit may include, in addition to the antibody or composition and the anti-fibrotic agent, other diagnostic or therapeutic agents. A kit can also include instructions for use in a diagnostic or therapeutic method.

### Gene Therapy

The antibodies used in the combination of the invention can be administered to a patient in need thereof via gene therapy. The therapy may be either in vivo or ex vivo. In a preferred embodiment, nucleic acid molecules encoding both a heavy chain and a light chain are administered to a patient. In a more preferred embodiment, the nucleic acid molecules are administered such that they are stably integrated into chromosomes of B cells because these cells are specialized for producing antibodies. In a preferred embodiment, precursor B cells are transfected or infected ex vivo and re-transplanted into a patient in need thereof. In another embodiment, precursor B cells or other cells are infected in vivo using a recombinant virus known to infect the cell type of interest. Typical vectors used for gene therapy include liposomes, plasmids and viral vectors. Exemplary viral vectors are retroviruses, adenoviruses and adeno-associated viruses. After infection either in vivo or ex vivo, levels of antibody expression can be monitored by taking a sample from the treated patient and using any immunoassay known in the art or discussed herein.

In a preferred embodiment, the gene therapy method comprises the steps of administering an isolated nucleic acid molecule encoding the heavy chain or an antigen-binding portion thereof of an anti-MAdCAM antibody and expressing the nucleic acid molecule. In another embodiment, the gene therapy method comprises the steps of administering an isolated nucleic acid molecule encoding the light chain or an antigen-binding portion thereof of an anti-MAdCAM antibody and expressing the nucleic acid molecule. In a more preferred method, the gene therapy method comprises the steps of administering of an isolated nucleic acid molecule encoding the heavy chain or an antigen-binding portion thereof and an isolated nucleic acid molecule encoding the light chain or the antigen-binding portion thereof of an anti-MAdCAM antibody of the invention and expressing the nucleic acid molecules. The gene therapy method may also comprise the step of administering another anti-inflammatory or immunomodulatory agent.

### Inhibition of α₄β₇/MAdCAM-Dependent Adhesion by Anti-MAdCAM Antibody:

The invention also provides the combination with an anti-fibrotic agent of an anti-MAdCAM antibody that binds MAdCAM and inhibits the binding and adhesion of α₄β₇-integrin bearing cells to MAdCAM or other cognate ligands, such as L-selectin, to MAdCAM. In a preferred embodiment, the MAdCAM is human and is either a soluble form, or expressed on the surface of a cell. In another preferred embodiment, the anti-MAdCAM antibody is a human antibody. In another embodiment, the antibody or portion thereof inhibits binding between α₄β₇ and MAdCAM with an IC₅₀ value of no more than 50 nM. In a preferred embodiment, the IC₅₀ value is no more than 5 nM. In a more preferred embodiment, the IC₅₀ value is less than 5 nM. In a more preferred embodiment, the IC₅₀ value is less than 0.05 μg/mL, 0.04 μg/mL or 0.03 μg/mL. In another preferred embodiment the IC₅₀ value is less than 0.5 μg/mL, 0.4 μg/mL or 0.3 μg/mL. The IC₅₀ value can be measured by any method known in the art. Typically, an IC₅₀ value can be measured by ELISA or adhesion assay. In a preferred embodiment, the IC₅₀ value is measured by adhesion assay using either cells or tissue which natively express MAdCAM or cells or tissue which have been engineered to express MAdCAM.

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology, immunology, microbiology, genetics, protein and nucleic acid chemistry and hybridization described herein are those well known and commonly used in the art. The methods and techniques of the present invention are generally performed according to conventional methods well known in the art and as described in various general and more specific references that are cited and discussed throughout the present specification unless otherwise indicated. See, e.g., Sambrook et al., Molecular Cloning: A Laboratory Manual, 2nd ed., Cold Spring Harbor Laboratory Press, Cold Spring Harbor, N.Y. (1989) and Ausubel et al., Current Protocols in Molecular Biology, Greene Publishing Associates (1992), and Harlow and Lane, Antibodies: A Laboratory Manual, Cold Spring Harbor Laboratory Press, Cold Spring Harbor, N.Y. (1990), which are incorporated herein by reference. Enzymatic reactions and purification techniques are performed according to manufacturer's specifications, as commonly accomplished in the art or as described herein. Standard techniques are used for chemical syntheses, chemical analyses, pharmaceutical preparation, formulation, and delivery, and treatment of patients.

The term “polypeptide” encompasses native or artificial proteins, protein fragments and polypeptide analogs of a protein sequence. A polypeptide may be monomeric or polymeric.

The term “isolated protein” or “isolated polypeptide” is a protein or polypeptide that by virtue of its origin or source of derivation (1) is not associated with naturally associated components that accompany it in its native state, (2) is free of other proteins from the same species (3) is expressed by a cell from a different species, or (4) does not occur in nature. Thus, a polypeptide that is chemically synthesized or synthesized in a cellular system different from the cell from which it naturally originates will be “isolated” from its naturally associated components. A protein may also be rendered substantially free of naturally associated components by isolation, using protein purification techniques well known in the art.

A protein or polypeptide is “substantially pure,” “substantially homogeneous” or “substantially purified” when at least about 60 to 75% of a sample exhibits a single species of polypeptide. The polypeptide or protein may be monomeric or multimeric. A substantially pure polypeptide or protein will typically comprise about 50%, 60%, 70%, 80% or 90% W/W of a protein sample, more usually about 95%, and preferably will be over 99% pure. Protein purity or homogeneity may be indicated by a number of means well known in the art, such as polyacrylamide gel electrophoresis of a protein sample, followed by visualizing a single polypeptide band upon staining the gel with a stain well known in the art. For certain purposes, higher resolution may be provided by using HPLC or other means well known in the art for purification.

The term “polypeptide fragment” as used herein refers to a polypeptide that has an amino-terminal and/or carboxy-terminal deletion, but where the remaining amino acid sequence is identical to the corresponding positions in the naturally-occurring sequence. In some embodiments, fragments are at least 5, 6, 8 or 10 amino acids long. In other embodiments, the fragments are at least 14 amino acids long, more preferably at least 20 amino acids long, usually at least 50 amino acids long, even more preferably at least 70, 80, 90, 100, 150 or 200 amino acids long.

The term “polypeptide analog” as used herein refers to a polypeptide that comprises a segment of at least 25 amino acids that has substantial identity to a portion of an amino acid sequence and that has at least one of the following properties: (1) specific binding to MAdCAM under suitable binding conditions, (2) ability to inhibit α₄β₇ integrin and/or L-selectin binding to MAdCAM, or (3) ability to reduce MAdCAM cell surface expression in vitro or in vivo. Typically, polypeptide analogs comprise a conservative amino acid substitution (or insertion or deletion) with respect to the naturally-occurring sequence. Analogs typically are at least 20 amino acids long, preferably at least 50, 60, 70, 80, 90, 100, 150 or 200 amino acids long or longer, and can often be as long as a full-length naturally-occurring polypeptide.

An “immunoglobulin” is a tetrameric molecule. In a naturally-occurring immunoglobulin, each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. Human light chains are classified as κ and λ light chains. Heavy chains are classified as μ, δ, γ, α, or ε, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 or more amino acids. See generally, Fundamental Immunology, Ch. 7 (Paul, W., ed., 2nd ed. Raven Press, N.Y. (1989)) (incorporated by reference in its entirety for all purposes). The variable regions of each light/heavy chain pair form the antibody binding site such that an intact immunoglobulin has two binding sites.

Immunoglobulin chains exhibit the same general structure of relatively conserved framework regions (FR) joined by three hypervariable regions, also called complementarity determining regions or CDRs. The CDRs from the two chains of each pair are aligned by the framework regions to form an epitope-specific binding site. From N-terminus to C-terminus, both light and heavy chains comprise the domains FR1, CDR1, FR2, CDR2, FR3, CDR3 and FR4. The assignment of amino acids to each domain is in accordance with the definitions of Kabat, Sequences of Proteins of Immunological Interest (National Institutes of Health, Bethesda, Md. (1987 and 1991)), or Chothia & Lesk, J. Mol. Biol., 196:901-917 (1987); Chothia et al., Nature, 342:878-883 (1989), each of which is incorporated herein by reference in their entirety.

An “antibody” refers to an intact immunoglobulin or to an antigen-binding portion thereof that competes with the intact antibody for specific binding. In some embodiments, an antibody is an antigen-binding portion thereof. Antigen-binding portions may be produced by recombinant DNA techniques or by enzymatic or chemical cleavage of intact antibodies. Antigen-binding portions include, inter alia, Fab, Fab′, F(ab′)₂, Fv, dAb, and complementarity determining region (CDR) fragments, single-chain antibodies (scFv), chimeric antibodies, diabodies and polypeptides that contain at least a portion of an immunoglobulin that is sufficient to confer specific antigen binding to the polypeptide. A Fab fragment is a monovalent fragment consisting of the VL, VH, CL and CH1 domains; a F(ab)₂ fragment is a bivalent fragment comprising two Fab fragments linked by a disulfide bridge at the hinge region; a Fd fragment consists of the VH and CH1 domains; an Fv fragment consists of the VL and VH domains of a single arm of an antibody; and a dAb fragment (Ward et al., Nature, 341:544-546 (1989)) consists of a VH domain.

As used herein, an antibody that is referred to as, e.g., 1.7.2, 1.8.2, 6.14.2, 6.34.2, 6.67.1, 6.77.2, 7.16.6, 7.20.5, 7.26.4 or 9.8.2, is a monoclonal antibody that is produced by the hybridoma of the same name. For example, antibody 1.7.2 is produced by hybridoma 1.7.2. An antibody that is referred to as 6.22.2-mod, 6.34.2-mod, 6.67.1-mod, 6.77.1-mod or 7.26.4-mod is a monoclonal antibody whose sequence has been modified from its corresponding parent by site-directed mutagenesis.

A single-chain antibody (scFv) is an antibody in which VL and VH regions are paired to form a monovalent molecule via a synthetic linker that enables them to be made as a single protein chain (Bird et al., Science, 242:423-426 (1988) and Huston et al., Proc. Natl. Acad. Sci. USA, 85:5879-5883 (1988)). Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see, e.g., Holliger, P., et al., Proc. Natl. Acad. Sci. USA, 90: 6444-6448 (1993) and Poljak, R. J., et al., Structure, 2:1121-1123 (1994)). One or more CDRs from an antibody of the invention may be incorporated into a molecule either covalently or noncovalently to make it an immunoadhesin that specifically binds to MAdCAM. An immunoadhesin may incorporate the CDR(s) as part of a larger polypeptide chain, may covalently link the CDR(s) to another polypeptide chain, or may incorporate the CDR(s) noncovalently. The CDRs permit the immunoadhesin to specifically bind to a particular antigen of interest.

An antibody may have one or more binding sites. If there is more than one binding site, the binding sites may be identical to one another or may be different. For instance, a naturally-occurring immunoglobulin has two identical binding sites, a single-chain antibody or Fab fragment has one binding site, while a “bispecific” or “bifunctional” antibody (diabody) has two different binding sites.

An “isolated antibody” is an antibody that (1) is not associated with naturally-associated components, including other naturally-associated antibodies, that accompany it in its native state, (2) is free of other proteins from the same species, (3) is expressed by a cell from a different species, or (4) does not occur in nature. Examples of isolated antibodies include an anti-MAdCAM antibody that has been affinity purified using MAdCAM, an anti-MAdCAM antibody that has been produced by a hybridoma or other cell line in vitro, and a human anti-MAdCAM antibody derived from a transgenic mammal or plant.

As used herein, the term “human antibody” means an antibody in which the variable and constant region sequences are human sequences. The term encompasses antibodies with sequences derived from human genes, but which have been changed, e.g., to decrease possible immunogenicity, increase affinity, eliminate cysteines or glycosylation sites that might cause undesirable folding, etc. The term encompasses such antibodies produced recombinantly in non-human cells which might impart glycosylation not typical of human cells. The term also emcompasses antibodies which have been raised in a transgenic mouse which comprises some or all of the human immunoglobulin heavy and light chain loci.

In one aspect, the invention provides a humanized antibody. In some embodiments, the humanized antibody is an antibody that is derived from a non-human species, in which certain amino acids in the framework and constant domains of the heavy and light chains have been mutated so as to avoid or abrogate an immune response in humans. In some embodiments, a humanized antibody may be produced by fusing the constant domains from a human antibody to the variable domains of a non-human species. Examples of how to make humanized antibodies may be found in U.S. Pat. Nos. 6,054,297, 5,886,152 and 5,877,293. In some embodiments, a humanized anti-MAdCAM antibody of the invention comprises the amino acid sequence of one or more framework regions of one or more human anti-MAdCAM antibodies of the invention.

In another aspect, the invention includes the use of a “chimeric antibody”. In some embodiments the chimeric antibody refers to an antibody that contains one or more regions from one antibody and one or more regions from one or more other antibodies. In a preferred embodiment, one or more of the CDRs are derived from a human anti-MAdCAM antibody of the invention. In a more preferred embodiment, all of the CDRs are derived from a human anti-MAdCAM antibody of the invention. In another preferred embodiment, the CDRs from more than one human anti-MAdCAM antibody of the invention are mixed and matched in a chimeric antibody. For instance, a chimeric antibody may comprise a CDR1 from the light chain of a first human anti-MAdCAM antibody may be combined with CDR2 and CDR3 from the light chain of a second human anti-MAdCAM antibody, and the CDRs from the heavy chain may be derived from a third anti-MAdCAM antibody. Further, the framework regions may be derived from one of the same anti-MAdCAM antibodies, from one or more different antibodies, such as a human antibody, or from a humanized antibody.

A “neutralizing antibody,” “an inhibitory antibody” or antagonist antibody is an antibody that inhibits the binding of α₄β₇ or α₄β₇-expressing cells, or any other cognate ligand or cognate ligand-expressing cells, to MAdCAM by at least about 20%. In a preferred embodiment, the antibody reduces inhibits the binding of α₄β₇ integrin or α₄β₇-expressing cells to MAdCAM by at least 40%, more preferably by 60%, even more preferably by 80%, 85%, 90%, 95% or 100%. The binding reduction may be measured by any means known to one of ordinary skill in the art, for example, as measured in an in vitro competitive binding assay. An example of measuring the reduction in binding of α₄β₇-expressing cells to MAdCAM is presented in Example I.

Fragments or analogs of antibodies can be readily prepared by those of ordinary skill in the art following the teachings of this specification. Preferred amino- and carboxy-termini of fragments or analogs occur near boundaries of functional domains. Structural and functional domains can be identified by comparison of the nucleotide and/or amino acid sequence data to public or proprietary sequence databases. Preferably, computerized comparison methods are used to identify sequence motifs or predicted protein conformation domains that occur in other proteins of known structure and/or function. Methods to identify protein sequences that fold into a known three-dimensional structure are known (Bowie et al., Science, 253:164 (1991)).

The term “k_{off}” refers to the off rate constant for dissociation of an antibody from the antibody/antigen complex.

The term “K_{d}” refers to the dissociation constant of a particular antibody-antigen interaction. An antibody is said to bind an antigen when the dissociation constant is ≦1 μM, preferably ≦100 nM and most preferably ≦10 nM.

The term “epitope” includes any protein determinant capable of specific binding to an immunoglobulin or T-cell receptor or otherwise interacting with a molecule. Epitopic determinants usually consist of chemically active surface groupings of molecules such as amino acids or carbohydrate side chains and usually have specific three dimensional structural characteristics, as well as specific charge characteristics. An epitope may be “linear” or “conformational.” In a linear epitope, all of the points of interaction between the protein and the interacting molecule (such as an antibody) occur linearally along the primary amino acid sequence of the protein. In a conformational epitope, the points of interaction occur across amino acid residues on the protein that are separated from one another.

As used herein, the twenty conventional amino acids and their abbreviations follow conventional usage. See Immunology—A Synthesis (2nd Edition, E. S. Golub and D. R. Gren, Eds., Sinauer Associates, Sunderland, Mass. (1991)), which is incorporated herein by reference. Stereoisomers (e.g., D-amino acids) of the twenty conventional amino acids, unnatural amino acids such as α-, α-disubstituted amino acids, N-alkyl amino acids, lactic acid, and other unconventional amino acids may also be suitable components for polypeptides of the present invention. Examples of unconventional amino acids include: 4-hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxylysine, s-N-methylarginine, and other similar amino acids and imino acids (e.g., 4-hydroxyproline). In the polypeptide notation used herein, the lefthand direction is the amino terminal direction and the righthand direction is the carboxy-terminal direction, in accordance with standard usage and convention.

The term “polynucleotide” as referred to herein means a polymeric form of nucleotides of at least 10 bases in length, either ribonucleotides or deoxynucleotides or a modified form of either type of nucleotide. The term includes single and double stranded forms of DNA.

The term “isolated polynucleotide” as used herein shall mean a polynucleotide of genomic, cDNA, or synthetic origin or some combination thereof, which by virtue of its origin the “isolated polynucleotide” (1) is not associated with all or a portion of a polynucleotide in which the “isolated polynucleotide” is found in nature, (2) is operably linked to a polynucleotide which it is not linked to in nature, or (3) does not occur in nature as part of a larger sequence.

The term “oligonucleotide” referred to herein includes naturally occurring, and modified nucleotides linked together by naturally occurring, and non-naturally occurring oligonucleotide linkages. Oligonucleotides are a polynucleotide subset generally comprising a length of 200 bases or fewer. Preferably oligonucleotides are 10 to 60 bases in length and most preferably 12, 13, 14, 15, 16, 17, 18, 19, or 20 to 40 bases in length. Oligonucleotides are usually single stranded, e.g., for probes; although oligonucleotides may be double stranded, e.g., for use in the construction of a gene mutant. Oligonucleotides of the invention can be either sense or antisense oligonucleotides.

The term “naturally occurring nucleotides” referred to herein includes deoxyribonucleotides and ribonucleotides. The term “modified nucleotides” referred to herein includes nucleotides with modified or substituted sugar groups and the like. The term “oligonucleotide linkages” referred to herein includes oligonucleotides linkages such as phosphorothioate, phosphorodithioate, phosphoroselenoate, phosphorodiselenoate, phosphoroanilothioate, phoshoraniladate, phosphoroamidate, and the like. See, e.g., LaPlanche et al., Nucl. Acids Res. 14:9081 (1986); Stec et al., J. Am. Chem. Soc. 106:6077 (1984); Stein et al., Nucl. Acids Res., 16:3209 (1988); Zon et al., Anti-Cancer Drug Design 6:539 (1991); Zon et al., Oligonucleotides and Analogues: A Practical Approach, pp. 87-108 (F. Eckstein, Ed., Oxford University Press, Oxford England (1991)); Stec et al., U.S. Pat. No. 5,151,510; Uhlmann and Peyman, Chemical Reviews, 90:543 (1990), the disclosures of which are hereby incorporated by reference. An oligonucleotide can include a label for detection, if desired.

“Operably linked” sequences include both expression control sequences that are contiguous with the gene of interest and expression control sequences that act in trans or at a distance to control the gene of interest. The term “expression control sequence” as used herein refers to polynucleotide sequences which are necessary to effect the expression and processing of coding sequences to which they are ligated. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (i.e., Kozak consensus sequence); sequences that enhance protein stability; and when desired, sequences that enhance protein secretion.

The nature of such control sequences differs depending upon the host organism; in prokaryotes, such control sequences generally include promoter, ribosomal binding site, and transcription termination sequence; in eukaryotes, generally, such control sequences include promoters and transcription termination sequence. The term “control sequences” is intended to include, at a minimum, all components whose presence is essential for expression and processing, and can also include additional components whose presence is advantageous, for example, leader sequences and fusion partner sequences.

The term “vector”, as used herein, is intended to refer to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The term “recombinant host cell” (or simply “host cell”), as used herein, is intended to refer to a cell into which a recombinant expression vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein.

The term “selectively hybridize” referred to herein means to detectably and specifically bind. Polynucleotides, oligonucleotides and fragments thereof in accordance with the invention selectively hybridize to nucleic acid strands under hybridization and wash conditions that minimize appreciable amounts of detectable binding to nonspecific nucleic acids. “High stringency” or “highly stringent” conditions can be used to achieve selective hybridization conditions as known in the art and discussed herein. An example of “high stringency” or “highly stringent” conditions is a method of incubating a polynucleotide with another polynucleotide, wherein one polynucleotide may be affixed to a solid surface such as a membrane, in a hybridization buffer of 6×SSPE or SSC, 50% formamide, 5×Denhardt's reagent, 0.5% SDS, 100 μg/ml denatured, fragmented salmon sperm DNA at a hybridization temperature of 42° C. for 12-16 hours, followed by twice washing at 55° C. using a wash buffer of 1×SSC, 0.5% SDS. See also Sambrook et al., supra, pp. 9.50-9.55.

The term “percent sequence identity” in the context of nucleotide sequences refers to the residues in two sequences which are the same when aligned for maximum correspondence. The length of sequence identity comparison may be over a stretch of at least about nine nucleotides, usually at least about 18 nucleotides, more usually at least about 24 nucleotides, typically at least about 28 nucleotides, more typically at least about 32 nucleotides, and preferably at least about 36, 48 or more nucleotides. There are a number of different algorithms known in the art which can be used to measure nucleotide sequence identity. For instance, polynucleotide sequences can be compared using FASTA, Gap or Bestfit, which are programs in Wisconsin Package Version 10.3, Accelrys, San Diego, Calif. FASTA, which includes, e.g., the programs FASTA2 and FASTA3, provides alignments and percent sequence identity of the regions of the best overlap between the query and search sequences (Pearson, Methods Enzymol., 183: 63-98 (1990); Pearson, Methods Mol. Biol., 132: 185-219 (2000); Pearson, Methods Enzymol., 266: 227-258 (1996); Pearson, J. Mol. Biol., 276: 71-84 (1998); herein incorporated by reference). Unless otherwise specified, default parameters for a particular program or algorithm are used. For instance, percent sequence identity between nucleotide sequences can be determined using FASTA with its default parameters (a word size of 6 and the NOPAM factor for the scoring matrix) or using Gap with its default parameters as provided in Wisconsin Package Version 10.3, herein incorporated by reference.

A reference to a nucleotide sequence encompasses its complement unless otherwise specified. Thus, a reference to a nucleic acid molecule having a particular sequence should be understood to encompass its complementary strand, with its complementary sequence.

In the molecular biology art, researchers use the terms “percent sequence identity”, “percent sequence similarity” and “percent sequence homology” interchangeably. In this application, these terms shall have the same meaning with respect to nucleotide sequences only.

The term “substantial similarity” or “substantial sequence similarity,” when referring to a nucleic acid or fragment thereof, indicates that, when optimally aligned with appropriate nucleotide insertions or deletions with another nucleic acid (or its complementary strand), there is nucleotide sequence identity in at least about 85%, preferably at least about 90%, and more preferably at least about 95%, 96%, 97%, 98% or 99% of the nucleotide bases, as measured by any well-known algorithm of sequence identity, such as FASTA, BLAST or Gap, as discussed above.

As applied to polypeptides, the term “substantial identity” means that two peptide sequences, when optimally aligned, such as by the programs GAP or BESTFIT using default gap weights, share at least 75% or 80% sequence identity, preferably at least 90% or 95% sequence identity, even more preferably at least 98% or 99% sequence identity. Preferably, residue positions that are not identical differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution. Means for making this adjustment are well-known to those of skill in the art. See, e.g., Pearson, Methods Mol. Biol., 24: 307-31 (1994), herein incorporated by reference. Examples of groups of amino acids that have side chains with similar chemical properties include 1) aliphatic side chains: glycine, alanine, valine, leucine and isoleucine; 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; and 6) sulfur-containing side chains are cysteine and methionine. Preferred conservative amino acids substitution groups are: valine-leucine-isoleucine, phenylalanine-tyrosine, lysine-arginine, alanine-valine, glutamate-aspartate, and asparagine-glutamine.

Alternatively, a conservative replacement is any change having a positive value in the PAM250 log-likelihood matrix disclosed in Gonnet et al., Science, 256: 1443-45 (1992), herein incorporated by reference. A “moderately conservative” replacement is any change having a nonnegative value in the PAM250 log-likelihood matrix.

Sequence similarity for polypeptides is typically measured using sequence analysis software. Protein analysis software matches similar sequences using measures of similarity assigned to various substitutions, deletions and other modifications, including conservative amino acid substitutions. For instance, GCG contains programs such as “Gap” and “Besffit” which can be used with default parameters to determine sequence homology or sequence identity between closely related polypeptides, such as homologous polypeptides from different species of organisms or between a wild type protein and a mutein thereof. See, e.g., Wisconsin package Version 10.3. Polypeptide sequences also can be compared using FASTA using default or recommended parameters, a program in Wisconsin package Version 10.3. FASTA (e.g., FASTA2 and FASTA3) provides alignments and percent sequence identity of the regions of the best overlap between the query and search sequences (Pearson (1990); Pearson (2000)). Another preferred algorithm when comparing a sequence of the invention to a database containing a large number of sequences from different organisms is the computer program BLAST, especially blastp or tblastn, using default parameters. See, e.g., Altschul et al., J. Mol. Biol. 215: 403-410 (1990); Altschul et al., Nucleic Acids Res. 25:3389-402 (1997); herein incorporated by reference.

The length of polypeptide sequences compared for homology will generally be at least about 16 amino acid residues, usually at least about 20 residues, more usually at least about 24 residues, typically at least about 28 residues, and preferably more than about 35 residues. When searching a database containing sequences from a large number of different organisms, it is preferable to compare amino acid sequences.

As used herein, the terms “label” or “labeled” refers to incorporation of another molecule in the antibody. In one embodiment, the label is a detectable marker, e.g., incorporation of a radiolabeled amino acid or attachment to a polypeptide of biotinyl moieties that can be detected by marked avidin (e.g., streptavidin containing a fluorescent marker or enzymatic activity that can be detected by optical or colorimetric methods). In another embodiment, the label or marker can be therapeutic, e.g., a drug conjugate or toxin. Various methods of labeling polypeptides and glycoproteins are known in the art and may be used. Examples of labels for polypeptides include, but are not limited to, the following: radioisotopes or radionuclides (e.g., ³H, ¹⁴C, ¹⁵N, ³⁵S, ⁹⁰Y, ⁹⁹Tc, ¹¹¹In, ¹²⁵I, ¹³¹I), fluorescent labels (e.g., FITC, rhodamine, lanthanide phosphors), enzymatic labels (e.g., horseradish peroxidase, β-galactosidase, luciferase, alkaline phosphatase), chemiluminescent markers, biotinyl groups, predetermined polypeptide epitopes recognized by a secondary reporter (e.g., leucine zipper pair sequences, binding sites for secondary antibodies, metal binding domains, epitope tags), magnetic agents, such as gadolinium chelates, toxins such as pertussis toxin, taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. In some embodiments, labels are attached by spacer arms of various lengths to reduce potential steric hindrance.

1. A combination of an anti-MAdCAM monoclonal antibody, or antigen binding portion thereof, and an anti-fibrotic agent.

2. The combination of claim 1 wherein the anti-fibrotic agent is a caspase inhibitor.

3. The combination of claim 2, wherein the caspase inhibitor is a compound of formula I: 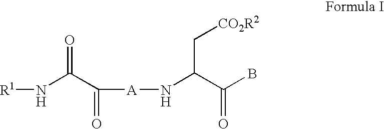
wherein
A is a natural or unnatural amino acid of Formula IIa-i: 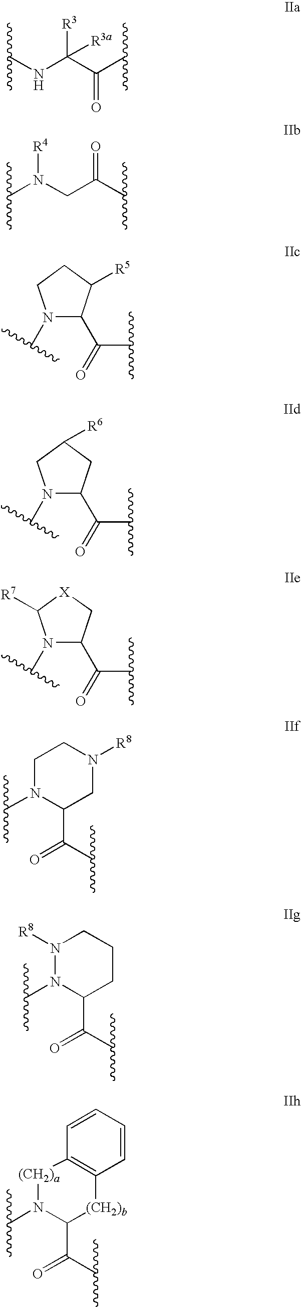 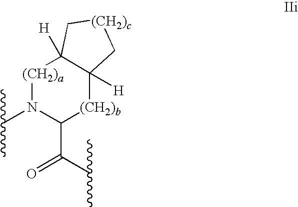
B is a hydrogen atom, a deuterium atom, alkyl, cycloalkyl, phenyl, substituted phenyl, naphthyl, substituted naphthyl, 2-benzoxazolyl, substituted 2-oxazolyl, (CH₂)ₙ-cycloalkyl, (CH₂)ₙ-phenyl, (CH₂)ₙ-(substituted phenyl), (CH₂)ₙ-(1 or 2-naphthyl), (CH₂)ₙ-(substituted 1 or 2-naphthyl), (CH₂)ₙ-(heteroaryl), (CH₂)ₙ-(substituted heteroaryl), halomethyl, CO₂R¹², CONR¹³R¹⁴, CH₂ZR¹⁵, CH₂OCO(aryl), CH₂OCO(heteroaryl), or CH₂OPO(R¹⁶)R¹⁷, where Z is an oxygen or a sulfur atom, or B is a group of the Formula IIIa-c: 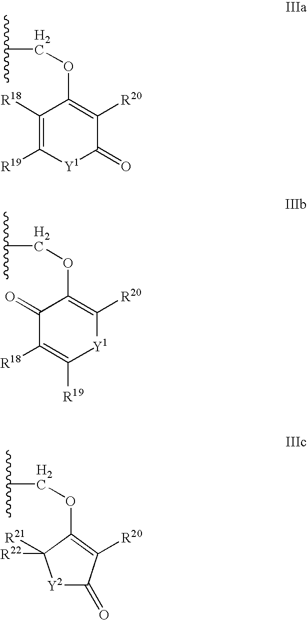
R¹ is alkyl, cycloalkyl, (cycloalkyl)alkyl, phenyl, substituted phenyl, phenylalkyl, substituted phenylalkyl, naphthyl, substituted naphthyl, (1 or 2 naphthyl)alkyl, substituted (1 or 2 naphthyl)alkyl, heteroaryl, substituted heteroaryl, (heteroaryl)alkyl, substituted (heteroaryl)alkyl, R^{1a}(R^{1b})N, or R^{1c}O; and
R² is hydrogen, lower alkyl, cycloalkyl, (cycloalkyl)alkyl, phenyl, substituted phenyl, phenylalkyl, substituted phenylalkyl, naphthyl, substituted naphthyl, (1 or 2 naphthyl)alkyl, or substituted (1 or 2 naphthyl)alkyl;
And wherein:
R^{1a} and R^{1b} are independently hydrogen, alkyl, cycloalkyl, (cycloalkyl)alkyl, phenyl, substituted phenyl, phenylalkyl, substituted phenylalkyl, naphthyl, substituted naphthyl, (1 or 2 naphthyl)alkyl, substituted (1 or 2 naphthyl)alkyl, heteroaryl, substituted heteroaryl, (heteroaryl)alkyl, or substituted (heteroaryl)alkyl, with the proviso that R^{1a} and R^{1b} cannot both be hydrogen;
R^{1c} is alkyl, cycloalkyl, (cycloalkyl)alkyl, phenyl, substituted phenyl, phenylalkyl, substituted phenylalkyl, naphthyl, substituted naphthyl, (1 or 2 naphthyl)alkyl, substituted (1 or 2 naphthyl)alkyl, heteroaryl, substituted heteroaryl, (heteroaryl)alkyl, or substituted (heteroaryl)alkyl;
R³ is C₁₋₆ alkyl, cycloalkyl, phenyl, substituted phenyl, (CH₂)ₙNH₂, (CH₂)NHCOR⁹, (CH₂)ₙN(C═NH)NH₂, (CH₂)ₘCO₂R², (CH₂)ₘOR¹⁰, (CH₂)ₘSR¹¹, (CH₂)ₙcycloalkyl, (CH₂)ₙphenyl, (CH₂)ₙ(substituted phenyl), (CH₂)ₙ(1 or 2-naphthyl) or (CH₂)ₙ(heteroaryl), wherein heteroaryl includes pyridyl, thienyl, furyl, thiazolyl, imidazolyl, pyrazolyl, isoxazolyl, pyrazinyl, pyrimidyl, triazinyl, tetrazolyl, and indolyl;
R^{3a} is hydrogen or methyl, or R³ and R^{3a} taken together are —(CH₂)_{d}— where d is an integer from 2 to 6;
R⁴ is phenyl, substituted phenyl, (CH₂)ₘphenyl, (CH₂)ₘ(substituted phenyl), cycloalkyl, or benzofused cycloalkyl;
R⁵ is hydrogen, lower alkyl, cycloalkyl, phenyl, substituted phenyl, (CH₂)ₙcycloalkyl, (CH₂)ₙphenyl, (CH₂)ₙ(substituted phenyl), or (CH₂)ₙ(1 or 2-naphthyl);
R⁶ is hydrogen, fluorine, oxo, lower alkyl, cycloalkyl, phenyl, substituted phenyl, naphthyl, (CH₂)ₙcycloalkyl, (CH₂)ₙphenyl, (CH₂)ₙ(substituted phenyl), (CH₂)ₙ(1 or 2-naphthyl), OR¹⁰, SR¹¹, or NHCOR⁹;
R⁷ is hydrogen, oxo (i.e. ═O), lower alkyl, cycloalkyl, phenyl, substituted phenyl, naphthyl, (CH₂)ₙcycloalkyl, (CH₂)ₙphenyl, (CH₂)ₙ(substituted phenyl), or (CH₂)ₙ(1 or 2-naphthyl);
R⁸ is lower alkyl, cycloalkyl, (CH₂)ₙcycloalkyl, (CH₂)ₙphenyl, (CH₂)ₙ(substituted phenyl), (CH₂)ₙ(1 or 2-naphthyl), or COR⁹;
R⁹ is hydrogen, lower alkyl, cycloalkyl, phenyl, substituted phenyl, naphthyl, (CH₂)ₙcycloalkyl, (CH₂)ₙphenyl, (CH₂)ₙ(substituted phenyl), (CH₂)ₙ(1 or 2-naphthyl), OR¹², or NR¹³R¹⁴;
R¹⁰ is hydrogen, lower alkyl, cycloalkyl, phenyl, substituted phenyl, naphthyl, (CH₂)ₙcycloalkyl, (CH₂)ₙphenyl, (CH₂)ₙ(substituted phenyl), or (CH₂)ₙ(1 or 2-naphthyl);
R¹¹ is lower alkyl, cycloalkyl, phenyl, substituted phenyl, naphthyl, (CH₂)ₙcycloalkyl, (CH₂)ₙphenyl, (CH₂)ₙ(substituted phenyl), or (CH₂)ₙ(1 or 2-naphthyl);
R¹² is lower alkyl, cycloalkyl, (CH₂)ₙcycloalkyl, (CH₂)ₙphenyl, (CH₂)ₙ(substituted phenyl), or (CH₂)ₙ(1 or 2-naphthyl);
R¹³ is hydrogen, lower alkyl, cycloalkyl, phenyl, substituted phenyl, naphthyl, substituted naphthyl, (CH₂)ₙcycloalkyl, (CH₂)ₙphenyl, (CH₂)ₙ(substituted phenyl), or (CH₂)ₙ(1 or 2-naphthyl);
R¹⁴ is hydrogen or lower alkyl; or
R¹³ and R¹⁴ taken together form a five to seven membered carbocyclic or heterocyclic ring, such as morpholine, or N-substituted piperazine;
R¹⁵ is phenyl, substituted phenyl, naphthyl, substituted naphthyl, heteroaryl, (CH₂)ₙphenyl, (CH₂)ₙ(substituted phenyl), (CH₂)ₙ(1 or 2-naphthyl), or (CH₂)ₙ(heteroaryl);
R¹⁶ or R¹⁷ are independently lower alkyl, cycloalkyl, phenyl, substituted phenyl, naphthyl, phenylalkyl, substituted phenylalkyl, or (cycloalkyl)alkyl;
R¹⁸ and R¹⁹ are independently hydrogen, alkyl, phenyl, substituted phenyl, (CH₂)ₙphenyl, (CH₂)ₙ(substituted phenyl), or R¹⁸ and R¹⁹ taken together are —(CH═CH)₂;
—R²⁰ is hydrogen, alkyl, phenyl, substituted phenyl, (CH₂)ₙphenyl, (CH₂)ₙ(substituted phenyl);
R²¹, R²² and R²³ are independently hydrogen, or alkyl;
X is CH₂, (CH₂)₂, (CH₂)₃, or S;
Y¹ is O or NR²³;
Y² is CH₂, O, or NR²³;
a is 0 or 1 and b is 1 or 2, provided that when a is 1 then b is 1;
c is 1 or 2, provided that when c is 1 then a is 0 and b is 1;
m is 1 or 2; and
n is 1, 2, 3, or 4;
or a pharmaceutically acceptable salt thereof.

4. The combination of claim 3, wherein the compound of formula I is selected from the group consisting of:
(3S)-3-[N—(N′-(2-Fluoro-4-Iodophenyl)Oxamyl)Valinyl]Amino-5-(2′,3′,5′,6′-Tetrafluorophenoxy)-4-Oxopentanoic acid;
(3S)-3-[N—(N′-(2-Chlorophenyl)Oxamyl)Valinyl]Amino-5-(2′,3′,5′,6′-Tetrafluorophenoxy)-4-Oxopentanoic acid;
(3S)-3-[N—(N′-(2-Bromophenyl)Oxamyl)Valinyl]Amino-5-(2′,3′,5′,6′-Tetrafluorophenoxy)-4-Oxopentanoic acid;
(3S)-3-[N—(N′-(2-Fluorophenyl)Oxamyl)Valinyl]Amino-5-(2′,3′,5′,6′-Tetrafluorophenoxy)-4-Oxopentanoic acid;
(3S)-3-[N—(N′-(2-Trifluoromethylphenyl)Oxamyl)Valinyl]Amino-5-(2′,3′,5′,6′-Tetrafluorophenoxy)-4-Oxopentanoic acid;
(3S)-3-[N—(N′-(1-Anthryl)Oxamyl)Valinyl]Amino-5-(2′,3′,5′,6′-Tetrafluorophenoxy)-4-Oxopentanoic acid;
(3S)-3-[N—(N′-(2-Tert-Butylphenyl)Oxamyl)Alaninyl]Amino-5-(2′,3′,5′,6′-Tetrafluorophenoxy)-4-Oxopentanoic acid;
(3S)-3-[N—(N′-(2-Trifluoromethylphenyl)Oxamyl)Alaninyl]Amino-5-(2′,3′,5′,6′-Tetrafluorophenoxy)-4-Oxopentanoic acid;
(3S)-3-[N—(N′-(2,6-Difluorophenyl)Oxamyl)Alaninyl]Amino-5-(2′,3′,5′,6′-Tetrafluorophenoxy)-4-Oxopentanoic acid;
(3S)-3-[N—(N′-(1-Naphthyl)Oxamyl)Alaninyl]Amino-5-(2′,3′,5′,6′-Tetrafluorophenoxy)-4-Oxopentanoic acid;
(3S)-3-[N—(N′-(4-Methoxyphenyl)Oxamyl)Alaninyl]Amino-5-(2′,3′,5′,6′-Tetrafluorophenoxy)-4-Oxopentanoic acid;
(3S)-3-[N—(N′-(2-Trifluoromethylphenyl)Oxamyl)Valinyl]Amino-4-Oxobutanoic acid;
(3S)-3-[N—(N′-(2-tert-Butylmethylphenyl)Oxamyl)Valinyl]Amino-4-Oxobutanoic acid;
(3S)-3-[N—(N′-(2-Benzylphenyl)Oxamyl)Valinyl]Amino-4-Oxobutanoic acid;
(3S)-3-[N—(N′-(2-Phenyl phenyl)Oxamyl)Valinyl]Amino-4-Oxobutanoic acid.

5. The combination of claim 1, wherein the anti-MAdCAM antibody, or antigen-binding portion, thereof is a human monoclonal antibody or antigen-binding portion.

6. The combination of claim 5, wherein the antibody, or portion, possesses at least one of the following properties:
(a) binds to human cells;
(b) has a selectivity for MAdCAM over VCAM or fibronectin of at least 100 fold;
(c) binds to human MAdCAM with a Kd of 3×10-¹⁰ M or less; or
(d) inhibits the binding of α₄β₇ expressing cells to human MAdCAM.
(e) inhibits the recruitment of lymphocytes to gastrointestinal lymphoid tissue.

7. The combination of claim 5, wherein said antibody, or antigen-binding portion, inhibits binding of human MAdCAM to α₄β₇, and wherein the antibody or portion thereof has at least one of the following properties:
(a) cross-competes with a reference antibody for binding to MAdCAM;
(b) competes with a reference antibody for binding to MAdCAM;
(c) binds to the same epitope of MAdCAM as a reference antibody;
(d) binds to MAdCAM with substantially the same K_{d} as a reference antibody;
(e) binds to MAdCAM with substantially the same off rate as a reference antibody;
wherein the reference antibody is selected from the group consisting of: monoclonal antibody 1.7.2, monoclonal antibody 1.8.2, monoclonal antibody 6.14.2, monoclonal antibody 6.22.2, monoclonal antibody 6.34.2, monoclonal antibody 6.67.1, monoclonal antibody 6.73.2, monoclonal antibody 6.77.1, monoclonal antibody 7.16.6, monoclonal antibody 7.20.5, monoclonal antibody 7.26.4, monoclonal antibody 9.8.2, monoclonal antibody 6.22.2-mod, monoclonal antibody 6.34.2-mod, monoclonal antibody 6.67.1-mod, monoclonal antibody 6.77.1-mod and monoclonal antibody 7.26.4-mod.

8. The combination of claim 5, wherein the monoclonal antibody, or an antigen-binding portion thereof, is selected from the following antibodies:
(a) the heavy chain comprises the heavy chain CDR¹, CDR² and CDR³ amino acid sequences of a reference antibody selected from the group consisting of: 1.7.2, 1.8.2, 6.14.2, 6.22.2, 6.34.2, 6.67.1, 6.73.2, 6.77.1, 7.16.6, 7.20.5, 7.26.4, 9.8.2, 6.22.2-mod, 6.34.2-mod, 6.67.1-mod, 6.77.1-mod and 7.26.4-mod
(b) the light chain comprises the light chain CDR1, CDR2 and CDR3 amino acid sequences of a reference antibody selected from the group consisting of: 1.7.2, 1.8.2, 6.14.2, 6.22.2, 6.34.2, 6.67.1, 6.73.2, 6.77.1, 7.16.6, 7.20.5, 7.26.4, 9.8.2, 6.22.2-mod, 6.34.2-mod, 6.67.1-mod, 6.77.1-mod and 7.26.4-mod
(c) the antibody comprises a heavy chain of (a) and a light chain of (b); and
(d) the antibody of (c) wherein the heavy chain and light chain CDR amino acid sequences are selected from the same reference antibody.

9. A combination of an anti-α₄β₇ integrin antibody, or antigen binding portion thereof, with a caspase inhibitor of claim 2.

10. A method for treating liver fibrosis, which method comprises administering to a patient in need thereof a therapeutically-effective amount of the combination of claim 1.

11. The method of claim 10, wherein the liver fibrosis is Hepatitic C-associated liver fibrosis, alcoholic liver disease or non-alcoholic steatohepatitis.

12. A pharmaceutical composition comprising the combination of claim 1, with a pharmaceutically acceptable carrier or excipient.


SEQUENCE LISTING (68 sequences)

SEQ ID NO: 1 - DNA, 1392 bp, Homo sapiens
atggagtttgggctgagctggattttccttgctgctattttaaaaggtgtccagtgtgaggtgcagctggtggagtctgggggaggcttggtgaagcctggggggtcccttagactctcctgtgtagcctctggattcactttcactaacgcctggatgatctgggtccgccaggctccagggaaggggctggagtgggttggccgtattaaaaggaaaactgatggtgggacaacagactacgctgcacccgtgaaaggcagattcaccatctcaagagatgattcaaaaaacacgctgtatctgcaaatgaacagcctgaaaaccgaggacacagccgtgtattactgtaccacagggggagtggctgaggactactggggccagggaaccctggtcaccgtctcctcagcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 2 - protein, 463 aa, Homo sapiens
MEFGLSWIFLAAILKGVQCEVQLVESGGGLVKPGGSLRLSCVASGFTFTNAWMIWVRQAPGKGLEWVGRIKRKTDGGTTDYAAPVKGRFTISRDDSKNTLYLQMNSLKTEDTAVYYCTTGGVAEDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 3 - DNA, 720 bp, Homo sapiens
atgaggctccctgctcagctcctggggctgctaatgctctgggtctctggatccagtggggatattgtgatgactcagtctccactctccctgcccgtcacccctggagagccggcctccatctcctgcaggtctagtcagagcctcctgcaaagtaatggatacaactatttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcaagctctacaaactatcaccttcggccaagggacacgactggagattaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagtga

SEQ ID NO: 4 - protein, 238 aa, Homo sapiens
MRLPAQLLGLLMLWVSGSSGDIVMTQSPLSLPVTPGEPASISCRSSQSLLQSNGYNYLDWYLQKPGQSPQLLIYLGSNRASGVPDRFSGSGSGTDFTLKISRVEAEDVGVYYCMQALQTITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - DNA, 1392 bp, Homo sapiens
atggagtttgggctgagctggattttccttgctgctattttaaaaggtgtccagtgtgaggtgcagctggtggagtctgggggaggcttggtgaagcctggggggtcccttagactctcctgtgtagtctctggattcactttcactaacgcctggatgatctgggtccgccaggctccagggaaggggctggagtgggttggccgtattaaaaggaaaactgatggtgggacaacagactacgctgcacccgtgaaaggcagattcaccatctcaagagatgattcaaaaaacacgctgtatctgcaaatgaacagcctgaaaaccgaggacacagccgtgtattactgtaccacagggggagtggctgaggactactggggccagggaaccctggtcaccgtctcctcagcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 6 - protein, 463 aa, Homo sapiens
MEFGLSWIFLAAILKGVQCEVQLVESGGGLVKPGGSLRLSCVVSGFTFTNAWMIWVRQAPGKGLEWVGRIKRKTDGGTTDYAAPVKGRFTISRDDSKNTLYLQMNSLKTEDTAVYYCTTGGVAEDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 7 - DNA, 720 bp, Homo sapiens
atgaggctccctgctcagctcctggggctgctaatgctctgggtctctggatccagtggggatattgtgatgactcagtctccactctccctgcccgtcacccctggagagccggcctccatctcctgcaggtctagtcagagcctcctgcaaagtaatggattcaactatttggattggtacctgcagaagccagggcagtctccacagctcctgatctatttgggttctaatcgggcctccggggtccctgacaggttcagtggcagtgggtcaggcacagattttacactgaaaatcagcagagtggaggctgaggatgttggggtttattactgcatgcaagctctacaaactatcaccttcggccaagggacacgactggagattaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagtga

SEQ ID NO: 8 - protein, 238 aa, Homo sapiens
MRLPAQLLGLLMLWVSGSSGDIVMTQSPLSLPVTPGEPASISCRSSQSLLQSNGFNYLDWYLQKPGQSPQLLIYLGSNRASGVPDRFSGSGSGTDFTLKISRVEAEDVGVYYCMQALQTITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - DNA, 1404 bp, Homo sapiens
atggagtttgggctgagctggctttttcttgtggctattttaaaaggtgtccagtgtgaggtgcagctgttggagtctgggggaggcttggtacagcctggggggtccctgagactctcctgtgcagcctctggactcacctttaacaattctgccatgacctgggtccgccaggctccagggaaggggctggagtgggtctcaactactagtggaagtggtggtaccacatactacgcagactccgtgaagggccggttcaccatctccagagactctcccaagaacacgctctatctgcaaatgaacagcctgagagccgaggacacggccgtatattactgtgcggcccgtggatacagctatggtacgaccccctatgagtactggggccagggaaccctggtcaccgtctcctcagcttccaccaagggcccatccgtcttccccctggcgccctgttccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaatga

SEQ ID NO: 10 - protein, 467 aa, Homo sapiens
MEFGLSWLFLVAILKGVQCEVQLLESGGGLVQPGGSLRLSCAASGLTFNNSAMTWVRQAPGKGLEWVSTTSGSGGTTYYADSVKGRFTISRDSPKNTLYLQMNSLRAEDTAVYYCAARGYSYGTTPYEYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - DNA, 711 bp, Homo sapiens
atggacatgagggtccccgctcagctcctggggctcctgctactctggctccgaggggccagatgtgacatccagatgacccagtctccatcctccctgtctgcatctgtaggagacagagtcaccatcacttgccgggcaagtcggagcattagcagctatttaaattggtatcagcagaaaccagggaaagcccctaaagtcctgatcttttttgtgtccagtttgcaaagtggggtcccatcaaggttcagtggcagtggctctgggacagatttcactctcaccatcagcagtctgcaacctgaagattttgcaacttactactgtcaacagaattacattccccctattaccttcggccaggggacacgactggagatcagacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 12 - protein, 236 aa, Homo sapiens
MDMRVPAQLLGLLLLWLRGARCDIQMTQSPSSLSASVGDRVTITCRASRSISSYLNWYQQKPGKAPKVLIFFVSSLQSGVPSRFSGSGSGTDFTLTISSLQPEDFATYYCQQNYIPPITFGQGTRLEIRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - DNA, 1398 bp, Homo sapiens
atggagtttgggctgagctgggttttcctcgttgctcttttaagaggtgtccagtgtcaggtgcagctggtggagtctgggggaggcgtggtccagcctgggaggtccctgagactctcctgtgcagcgtctggacacaccttcagtagcgatggcatgcactgggtccgccaggctccaggcaaggggctggagtgggtggcaattatatggtatgatggaagtaataaatattatgcagactccgtgaagggccgattcaccatctccagagacaattccaagaacacgctgtatctgcaaatgaacagcctgagagccgaggacacggctgtatattactgtgcgagagatcccggctactattacggtatggacgtctggggccaagggaccacggtcaccgtctcctcagcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccgcgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaatga

SEQ ID NO: 14 - protein, 465 aa, Homo sapiens
MEFGLSWVFLVALLRGVQCQVQLVESGGGVVQPGRSLRLSCAASGHTFSSDGMHWVRQAPGKGLEWVAIIWYDGSNKYYADSVKGRFTISRDNSKNTLYLQMNSLRAEDTAVYYCARDPGYYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTAPPVLDSDGSFFLYSRLTVDKSRWQEGNVFSCSVMHEALHNHYTQKSLSLSLGK

SEQ ID NO: 15 - DNA, 705 bp, Homo sapiens
atgttgccatcacaactcattgggtttctgctgctctgggttccagcttccaggggtgaaattgtgctgactcagtctccagactttcagtctgtgactccaaaagagaaagtcaccatcacctgccgggccagtcagagaattggtagtagcttacactggtaccagcagaaaccagatcagtctccaaaactcctcatcaagtatgcttcccagtccttctcaggggtcccctcgaggttcagtggcagtggatctgggacaaatttcaccctcaccatcaatggcctggaagctgaagatgctgcaacttattactgtcatcagagtggtcgtttaccgctcactttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagtga

SEQ ID NO: 16 - protein, 233 aa, Homo sapiens
MLPSQLIGFLLLWVPASRGEIVLTQSPDFQSVTPKEKVTITCRASQRIGSSLHWYQQKPDQSPKLLIKYASQSFSGVPSRFSGSGSGTNFTLTINGLEAEDAATYYCHQSGRLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - DNA, 1410 bp, Homo sapiens
atggagtttgggctgagctgggttttcctcgttgctcttttaagaggtgtccagtgtcaggtgcagctggtggagtctgggggaggcgtggtccagcctgggaggtccctgagactctcctgtgcagcctctggattcaccttcagtagctatggcatgcactgggtccgccaggctccaggcaaggggctggagtgggtggcagttatatcaaatgatggaaataataaatactatgcagactccgtgaagggccgattcaccatctccagagacaattccaaaaacacgctgtatctgcaaatgaacagcctgagcgctgaggacacggctgtgtattactgtgcgagagatagtacggcgataacctactactactacggaatggacgtctggggccaagggaccacggtcaccgtctcctcagcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatggacagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaatga

SEQ ID NO: 18 - protein, 469 aa, Homo sapiens
MEFGLSWVFLVALLRGVQCQVQLVESGGGVVQPGRSLRLSCAASGFTFSSYGMHWVRQAPGKGLEWVAVISNDGNNKYYADSVKGRFTISRDNSKNTLYLQMNSLSAEDTAVYYCARDSTAITYYYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 19 - DNA, 714 bp, Homo sapiens
atggacatgagggtccccgctcagctcctggggctcctgctactctggctccgaggtgccagatgtgacatccagatgacccagtctccatcctccctgtctgcatctgtcggagacagagtcaccatcacttgccgggcaagtcagaatattagtagctatttaaattggtttcagcagaaaccagggaaagcccctaagctcctgatctatgctgcatccggtttgaagcgtggggtcccatcacggttcagtggtagtggatctgggacagatttcactctcaccatcaggactctgcaacctgatgattttgcaacttactcctgtcaccagagttacagtctcccattcactttcggccctgggaccaaagtggatatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagtga

SEQ ID NO: 20 - protein, 236 aa, Homo sapiens
MDMRVPAQLLGLLLLWLRGARCDIQMTQSPSSLSASVGDRVTITCRASQNISSYLNWFQQKPGKAPKLLIYAASGLKRGVPSRFSGSGSGTDFTLTIRTLQPDDFATYSCHQSYSLPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - DNA, 1413 bp, Homo sapiens
atgaaacacctgtggttcttcctcctgctggtggcagctcccagatgggtcctgtcccaggtgcagctgcaggagtcgggcccaggactggtgaagccttcggagaccctgtccctcacctgcactgtctctggtgactccatcagtagtaactattggagctggatccggcagcccgccgggaagggactggagtggattgggcgtatctataccagtgggggcaccaactccaacccctccctcaggggtcgagtcaccattttagcagacacgtccaagaaccagttctctctgaaactgagttctgtgaccgccgcggacacggccgtgtattactgtgcgagagatcgtattactataattcggggacttattccatccttctttgactactggggccagggaaccctggtcaccgtctcctcagcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaatga

SEQ ID NO: 22 - protein, 470 aa, Homo sapiens
MKHLWFFLLLVAAPRWVLSQVQLQESGPGLVKPSETLSLTCTVSGDSISSNYWSWIRQPAGKGLEWIGRIYTSGGTNSNPSLRGRVTILADTSKNQFSLKLSSVTAADTAVYYCARDRITIIRGLIPSFFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 23 - DNA, 729 bp, Homo sapiens
atggtgttgcagacccaggtcttcatttctctgttgctctggatctctggtgcctacggggacatcgtgatgacccagtctccagactccctggctgtgtctctgggcgagagggccaccatcaactgcaagtccagccagagtgttttatacagctccaacaataagacctacttagcttggtaccaacagaaaccaagacagcctcctaaattgctcatttactgggcatctatacgggaatatggggtccctgaccgattcagtggcagcgggtctgggacagatttcactctcaccatcagcagcctgcaggctgaagatgtggcagtttatttctgtcaacaatattatagtattcctcccctcactttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagtga

SEQ ID NO: 24 - protein, 241 aa, Homo sapiens
MVLQTQVFISLLLWISGAYGDIVMTQSPDSLAVSLGERATINCKSSQSVLYSSNNKTYLAWYQQKPRQPPKLLIYWASIREYGVPDRFSGSGSGTDFTLTISSLQAEDVAVYFCQQYYSIPPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - DNA, 1419 bp, Homo sapiens
atggagtttgggctgagctggctttttcttgtggctattttaaaaggtgtccagtgtgaggtgcagctgttggagtctgggggagacttggtccagcctggggggtccctgagactctcctgtgcagcctctggattcacctttagaagttatgccatgaactgggtccgacaggctccagggaaggggctggagtgggtctcagttattagtggtcgtggtggtactacatactacgcagactccgtgaagggccggttcaccatctccagagacaattccaagaacacgctgtatctgcaaatgaacagcctgagagccgaggacgcggccgtatattactgtgcgaagatagcagtggctggagaggggctctactactactacggtatggacgtctggggccaagggaccacggtcaccgtctcctcagcttccaccaagggcccatccgtcttccccctggcgccctgctccaggagcacctccgagaacacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctctagcagcttgggcacgaagacctacacctgcaacgtagatcacaagcccagcaacaccaaggtggacaagagagttgagtccaaatatggtcccccatgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaatgatag

SEQ ID NO: 26 - protein, 471 aa, Homo sapiens
MEFGLSWLFLVAILKGVQCEVQLLESGGDLVQPGGSLRLSCAASGFTFRSYAMNWVRQAPGKGLEWVSVISGRGGTTYYADSVKGRFTISRDNSKNTLYLQMNSLRAEDAAVYYCAKIAVAGEGLYYYYGMDVWGQGTTVTVSSASTKGPSVFPLAPCSRSTSENTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 27 - DNA, 720 bp, Homo sapiens
atggacatgagggtccccgctcagctcctggggctcctgctactctggctccgaggtgccagatgtgacatccagatgacccagtctccatcctccctgtctgcatctgtaggtgacagagtcaccttcacttgccgggcaagtcagaacattaccaactatttaaattggtatcagcagaaaccagggaaggcccctaagctcctgatctatgctgcgtccagtttgccaagaggggtcccatcaaggttccgtggcagtggatctgggacagatttcactctcaccatcagcagtctgcaacctgaagattttgcaacttactactgtcaacagagttacagtaatcctccggagtgcggttttggccaggggaccacgctggatatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagtga

SEQ ID NO: 28 - protein, 238 aa, Homo sapiens
MDMRVPAQLLGLLLLWLRGARCDIQMTQSPSSLSASVGDRVTFTCRASQNITNYLNWYQQKPGKAPKLLIYAASSLPRGVPSRFRGSGSGTDFTLTISSLQPEDFATYYCQQSYSNPPECGFGQGTTLDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - DNA, 1434 bp, Homo sapiens
atggaactggggctccgctgggttttccttgttgctattttagaaggtgtccagtgtgaggtgcagctggtggagtctgggggaggcctggtcaagcctggggggtccctgagactctcctgtgcagcctctggattcaccttcagtagctatagcatgaactgggtccgccaggctccagggaaggggctggagtgggtctcatccattagtagtagtagtagttacatatactacgcagactcagtgaagggccgattcaccatctccagagacaacgccaagaactcactgtatctgcaaatgaacagcctgagagccgaggacacggctgtgtattactgtgcgagagatgggtatagcagtggctggtcctactactactactacggtatggacgtctggggccaagggaccacggtcaccgtctcctcagcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttttcacgctccgtgatgcatgaggctctgcacaaccactacacacagaagagcctctccctgtctctgggtaaatgataggaattctgatga

SEQ ID NO: 30 - protein, 472 aa, Homo sapiens
MELGLRWVFLVAILEGVQCEVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARDGYSSGWSYYYYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSRSVMHEALHNHYTQKSLSLSLGK

SEQ ID NO: 31 - DNA, 723 bp, Homo sapiens
atgaggctccctgctcagctcctggggctgctaatgctctggatacctggatccagtgcagatattgtgatgacccagactccactctctctgtccgtcactcctggacagccggcctccatctcctgcaactctagtcagagcctcctgcttagtgatggaaagacctatttgaattggtacctgcagaagcccggccagcctccacagctcctgatctatgaagtttccaaccggttctctggagtgccagacaggttcagtggcagcgggtcagggacagatttcacactgaaaatcagccgggtggaggctgaggatgttggggtttattcctgcatgcaaagtatacagcttatgtgcagttttggccaggggaccaagctggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagtga

SEQ ID NO: 32 - protein, 239 aa, Homo sapiens
MRLPAQLLGLLMLWIPGSSADIVMTQTPLSLSVTPGQPASISCNSSQSLLLSDGKTYLNWYLQKPGQPPQLLIYEVSNRFSGVPDRFSGSGSGTDFTLKISRVEAEDVGVYSCMQSIQLMCSFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 33 - DNA, 1410 bp, Homo sapiens
atggactggacctggagcatccttttcttggtggcagcagcaacaggtgcccactcccaggttcagctggtgcagtctggagctgaggtgaagaagcctggggcctcagtgaaggtctcctgcaaggcttctggttacacctttaccagctatggtatcaactgggtgcgacaggcccctggacaagggcttgagtggatgggatggatcagcgtttacagtggtaacacaaactatgcacagaaggtccagggcagagtcaccatgaccgcagacacatccacgagcacagcctacatggacctgaggagcctgagatctgacgacacggccgtgtattactgtgcgagagagggtagcagctcgtccggagactactattacggtatggacgtctggggccaagggaccacggtcaccgtctcctcagcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 34 - protein, 469 aa, Homo sapiens
MDWTWSILFLVAAATGAHSQVQLVQSGAEVKKPGASVKVSCKASGYTFTSYGINWVRQAPGQGLEWMGWISVYSGNTNYAQKVQGRVTMTADTSTSTAYMDLRSLRSDDTAVYYCAREGSSSSGDYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 35 - DNA, 723 bp, Homo sapiens
atgaggctccctgctcagctcctggggctgctaatgctctggatacctggatccagtgcagatattgtgatgacccagactccactctctctgtccgtcacccctggacagccggcctccatctcctgcaagtctagtcagagcctcctgcatactgatggaacgacctatttgtattggtacctgcagaagccaggccagcctccacagctcctgatctatgaagtttccaaccggttctctggagtgccagataggttcagtggcagcgggtcagggacagatttcacactgaaaatcagccgggtggaggctgaggatgttgggatttattactgcatgcaaaatatacagcttccgtggacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagtga

SEQ ID NO: 36 - protein, 239 aa, Homo sapiens
MRLPAQLLGLLMLWIPGSSADIVMTQTPLSLSVTPGQPASISCKSSQSLLHTDGTTYLYWYLQKPGQPPQLLIYEVSNRFSGVPDRFSGSGSGTDFTLKISRVEAEDVGIYYCMQNIQLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 37 - DNA, 1416 bp, Homo sapiens
atgaaacacctgtggttcttcctcctgctggtggcagctcccagatgggtcctgtcccaggtgcagctgcaggagtcgggcccaggactggtgaagccttcggagaccctgtccctcacctgcactgtctctggtagctccatcagtagttaccactggaactggatccggcagcccgccgggaagggactggagtggattgggcgtatctataccagtgggagcaccaactacaacccctccctcaagagtcgagtcaccatgtcactagacacgtccaagaaccagttctccctgaagctgagctctgtgaccgccgcggacacggccgtgtattactgtgcgagagagggggtcaggtattactatgcttcggggagttattactacggtctggacgtctggggccaagggaccacggtcaccgtctcctcagcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 38 - protein, 471 aa, Homo sapiens [sequence omitted]

SEQ ID NO: 39 - DNA, 720 bp, Homo sapiens
atgaggctccctgctcagctcctggggctgctaatgctctgggtctctggatccagtggggatattgtgatgactcagtctccactctccctgcccgtcacccctggagagccggcctccatctcctgcaggtctagtcagagcctcctgcatggtaatggatacaactatttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcaagctctacaaactctcactttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagtga

SEQ ID NO: 40 - protein, 238 aa, Homo sapiens
MRLPAQLLGLLMLWVSGSSGDIVMTQSPLSLPVTPGEPASISCRSSQSLLHGNGYNYLDWYLQKPGQSPQLLIYLGSNRASGVPDRFSGSGSGTDFTLKISRVEAEDVGVYYCMQALQTLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - DNA, 1410 bp, Homo sapiens
atggactggacctggagcatccttttcttggtggcagcagcaacaggtgcccactcccaggttcagctggtgcagtctggagctgaggtgaagaagcctggggcctcagtgaaggtctcctgcgaggcttctggttacacctttaccagctatggtatcgactgggtgcgacaggcccctggacaagggcttgagtggatgggatggatcagcgtttacagtggtaacacaaactatgcacagaagctccagggcagagtcaccatgtccacagacacatccacgagcacagcctacatggagctgaggagcctgagatctgacgacacggccgtgtattactgtgcgagagagggtagcagctcgtccggagactactactacggtatggacgtctggggccaagggaccacggtcaccgtctcctcagcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccattgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 42 - protein, 469 aa, Homo sapiens
MDWTWSILFLVAAATGAHSQVQLVQSGAEVKKPGASVKVSCEASGYTFTSYGIDWVRQAPGQGLEWMGWISVYSGNTNYAQKLQGRVTMSTDTSTSTAYMELRSLRSDDTAVYYCAREGSSSSGDYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 43 - DNA, 723 bp, Homo sapiens
atgaggctccctgctcagctcctggggctgctaatgctctggatacctggatccagtgcggatattgtgatgacccagactccactctctctgtccgtcacccctggacagccggcctccatctcctgcaagtctaatcagagcctcctgtatagtgatggaaagacctatttgttttggtacctgcagaagccaggccagcctccacagctcctgatctatgaagtttccaaccgattctctggagtgccagataggttcagtggcagcgggtcagggacagatttcacactgaaaatcagccgggtggaggctgaggatgttggggtttattactgcatgcaaagtatacagcttccgtggacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagtga

SEQ ID NO: 44 - protein, 239 aa, Homo sapiens
MRLPAQLLGLLMLWIPGSSADIVMTQTPLSLSVTPGQPASISCKSNQSLLYSDGKTYLFWYLQKPGQPPQLLIYEVSNRFSGVPDRFSGSGSGTDFTLKISRVEAEDVGVYYCMQSIQLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 45 - DNA, 1389 bp, Homo sapiens
atggagtttgggctgagctgggttttcctcgttgctcttttaagaggtgtccagtgtcaggtgcagctggtggagtctgggggaggcgtggtccagcctgggaggtccctgagactctcctgtgcagcgtctggattcaccttcagtagctatggcatgcactgggtccgccaggctccaggcaaggggctggagtgggtggcagttatatggtatgatggaagtaatgaatactatgcagactccgtgaagggccgattcaccatctccagagacaattccaagaacacgctgtatctgcaaatgaacagcctgagagccgaggacacggctgtgtattactgtgcgaggggggcgtaccactttgcctactggggccagggaaccctggtcaccgtctcctcagcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaatga

SEQ ID NO: 46 - protein, 462 aa, Homo sapiens
MEFGLSWVFLVALLRGVQCQVQLVESGGGVVQPGRSLRLSCAASGFTFSSYGMHWVRQAPGKGLEWVAVIWYDGSNEYYADSVKGRFTISRDNSKNTLYLQMNSLRAEDTAVYYCARGAYHFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 47 - DNA, 720 bp, Homo sapiens
atggacatgagggtccctgctcagctcctggggctcctgctgctctggctctcagtcgcaggtgccagatgtgacatccagatgacccagtctccatcctccctgtctgcatctgtaggagacagagtcaccatcacttgccaggcgagtcaggacattagcaactatttaaattggtatcagcagaaaccagggaaagcccctaagctcctgatctacgatgcatccaatttggaaacaggggtcccatcaaggttcagtggaagtggatctgggacagattttactttcaccatcagcagcctgcagcctgaagatattgcaacatattcctgtcaacactctgataatctctcgatcaccttcggccaggggacacgactggagattaaacgaactgtggctgcaccatctgtcttcatcttcccgccatctgatgagcagttgaaatctggaactgcctctgttgtgtgcctgctgaataacttctaccccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagtga

SEQ ID NO: 48 - protein, 238 aa, Homo sapiens
MDMRVPAQLLGLLLLWLSVAGARCDIQMTQSPSSLSASVGDRVTITCQASQDISNYLNWYQQKPGKAPKLLIYDASNLETGVPSRFSGSGSGTDFTFTISSLQPEDIATYSCQHSDNLSITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 49 - DNA, 681 bp, Macaca fascicularis
atggatcggggcctggccctcctgctggcggggcttctggggctcctccagccgggctgcggccagtccctccaggtgaagcccctgcaggtggagcccccggagccggtggtggccgtggccctgggcgcctctcgccagctcacctgccgcctggactgcgcggacggcggggccacggtgcagtggcggggcctggacaccagcctgggcgcggtgcagtcggacgcgggccgcagcgtcctcaccgtgcgcaacgcctcgctgtcggcggccgggacccgtgtgtgcgtgggctcctgcgggggccgcaccttccagcacaccgtgcggctccttgtgtacgccttcccggaccagctgaccatctccccggcagccctggtgcctggtgacccggaggtggcctgtacggctcacaaagtcacgcctgtggaccccaatgcgctctccttctccctgctcctgggggaccaggaactggagggggcccaggctctgggcccggaggtggaggaggaggaggagccccaggaggaggaggacgtgctgttcagggtgacagagcgctggcggctgccgaccctggcaacccctgtcctgcccgcgctctactgccaggccacgatgaggctgcctggcttggagctcagccaccgccaggccatcccggtcctgcac

SEQ ID NO: 50 - protein, 227 aa, Macaca fascicularis
MDRGLALLLAGLLGLLQPGCGQSLQVKPLQVEPPEPVVAVALGASRQLTCRLDCADGGATVQWRGLDTSLGAVQSDAGRSVLTVRNASLSAAGTRVCVGSCGGRTFQHTVRLLVYAFPDQLTISPAALVPGDPEVACTAHKVTPVDPNALSFSLLLGDQELEGAQALGPEVEEEEEPQEEEDVLFRVTERWRLPTLATPVLPALYCQATMRLPGLELSHRQAIPVLH

SEQ ID NO: 51 - DNA, 1398 bp, Homo sapiens
atggagtttgggctgagctgggttttcctcgttgctcttttaagaggtgtccagtgtcaggtgcagctggtggagtctgggggaggcgtggtccagcctgggaggtccctgagactctcctgtgcagcgtctggattcaccttcagtagcgatggcatgcactgggtccgccaggctccaggcaaggggctggagtgggtggcaattatatggtatgatggaagtaataaatattatgcagactccgtgaagggccgattcaccatctccagagacaattccaagaacacgctgtatctgcaaatgaacagcctgagagccgaggacacggctgtatattactgtgcgagagatcccggctactattacggtatggacgtctggggccaagggaccacggtcaccgtctcctcagcttccaccaagggcccatccgtcttccccctggcgccctgctctagaagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgatag

SEQ ID NO: 52 - protein, 464 aa, Homo sapiens
MEFGLSWVFLVALLRGVQCQVQLVESGGGVVQPGRSLRLSCAASGFTFSSDGMHWVRQAPGKGLEWVAIIWYDGSNKYYADSVKGRFTISRDNSKNTLYLQMNSLRAEDTAVYYCARDPGYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 53 - DNA, 705 bp, Homo sapiens
atgttgccatcacaactcattgggtttctgctgctctgggttccagcttccaggggtgaaattgtgctgactcagtctccagactttcagtctgtgactccaaaagagaaagtcaccatcacctgccgggccagtcagagaattggtagtagcttacactggtaccagcagaaaccagatcagtctccaaaactcctcatcaagtatgcttcccagtccttctcaggggtcccctcgaggttcagtggcagtggatctgggacagatttcaccctcaccatcaatagcctggaagctgaagatgctgcaacttattactgtcatcagagtggtcgtttaccgctcactttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagtga

SEQ ID NO: 54 - protein, 233 aa, Homo sapiens
MLPSQLIGFLLLWVPASRGEIVLTQSPDFQSVTPKEKVTITCRASQRIGSSLHWYQQKPDQSPKLLIKYASQSFSGVPSRFSGSGSGTDFTLTINSLEAEDAATYYCHQSGRLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 55 - DNA, 1410 bp, Homo sapiens
atggagtttgggctgagctgggttttcctcgttgctcttttaagaggtgtccagtgtcaggtgcagctggtggagtctgggggaggcgtggtccagcctgggaggtccctgagactctcctgtgcagcctctggattcaccttcagtagctatggcatgcactgggtccgccaggctccaggcaaggggctggagtgggtggcagttatatcaaatgatggaaataataaatactatgcagactccgtgaagggccgattcaccatctccagagacaattccaaaaacacgctgtatctgcaaatgaacagcctgcgcgctgaggacacggctgtgtattactgtgcgagagatagtacggcgataacctactactactacggaatggacgtctggggccaagggaccacggtcaccgtctcctcagcttccaccaagggcccatccgtcttccccctggcgccctgctctagaagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgatag

SEQ ID NO: 56 - protein, 468 aa, Homo sapiens
MEFGLSWVFLVALLRGVQCQVQLVESGGGVVQPGRSLRLSCAASGFTFSSYGMHWVRQAPGKGLEWVAVISNDGNNKYYADSVKGRFTISRDNSKNTLYLQMNSLRAEDTAVYYCARDSTAITYYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 57 - DNA, 714 bp, Homo sapiens
atggacatgagggtccccgctcagctcctggggctcctgctactctggctccgaggtgccagatgtgacatccagatgacccagtctccatcctccctgtctgcatctgtcggagacagagtcaccatcacttgccgggcaagtcagagtattagtagctatttaaattggtatcagcagaaaccagggaaagcccctaagctcctgatctatgctgcatccggtttgaagcgtggggtcccatcacggttcagtggtagtggatctgggacagatttcactctcaccatcagttctctgcaacctgaggattttgcaacttactactgtcaccagagttacagtctcccattcactttcggccctgggaccaaagtggatatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagtga

SEQ ID NO: 58 - protein, 236 aa, Homo sapiens
MDMRVPAQLLGLLLLWLRGARCDIQMTQSPSSLSASVGDRVTITCRASQSISSYLNWYQQKPGKAPKLLIYAASGLKRGVPSRFSGSGSGTDFTLTISSLQPEDFATYYCHQSYSLPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 59 - DNA, 1413 bp, Homo sapiens
atgaaacacctgtggttcttcctcctgctggtggcagctcccagatgggtcctgtcccaggtgcagctgcaggagtcgggcccaggactggtgaagccttcggagaccctgtccctcacctgcactgtctctggtgactccatcagtagtaactattggagctggatccggcagcccgccgggaagggactggagtggattgggcgtatctataccagtgggggcaccaactccaacccctccctcaggggtcgagtcaccatgtcagtagacacgtccaagaaccagttctctctgaaactgagttctgtgaccgccgcggacacggccgtgtattactgtgcgagagatcgtattactataattcggggacttattccatccttctttgactactggggccagggaaccctggtcaccgtctcctcagcttccaccaagggcccatccgtcttccccctggcgccctgctctagaagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgatag

SEQ ID NO: 60 - protein, 469 aa, Homo sapiens
MKHLWFFLLLVAAPRWVLSQVQLQESGPGLVKPSETLSLTCTVSGDSISSNYWSWIRQPAGKGLEWIGRIYTSGGTNSNPSLRGRVTMSVDTSKNQFSLKLSSVTAADTAVYYCARDRITIIRGLIPSFFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 61 - DNA, 729 bp, Homo sapiens
atggtgttgcagacccaggtcttcatttctctgttgctctggatctctggtgcctacggggacatcgtgatgacccagtctccagactccctggctgtgtctctgggcgagagggccaccatcaactgcaagtccagccagagtgttttatacagctccaacaataagaactacttagcttggtaccaacagaaaccaggacagcctcctaaattgctcatttactgggcatctatacgggaatatggggtccctgaccgattcagtggcagcgggtctgggacagatttcactctcaccatcagcagcctgcaggctgaagatgtggcagtttatttctgtcaacaatattatagtattcctcccctcactttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagtga

SEQ ID NO: 62 - protein, 241 aa, Homo sapiens
MVLQTQVFISLLLWISGAYGDIVMTQSPDSLAVSLGERATINCKSSQSVLYSSNNKNYLAWYQQKPGQPPKLLIYWASIREYGVPDRFSGSGSGTDFTLTISSLQAEDVAVYFCQQYYSIPPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 63 - DNA, 1419 bp, Homo sapiens
atggaactggggctccgctgggttttccttgttgctattttagaaggtgtccagtgtgaggtgcagctggtggagtctgggggaggcctggtcaagcctggggggtccctgagactctcctgtgcagcctctggattcaccttcagtagctatagcatgaactgggtccgccaggctccagggaaggggctggagtgggtctcatccattagtagtagtagtagttacatatactacgcagactcagtgaagggccgattcaccatctccagagacaacgccaagaactcactgtatctgcaaatgaacagcctgagagccgaggacacggctgtgtattactgtgcgagagatgggtatagcagtggctggtcctactactactactacggtatggacgtctggggccaagggaccacggtcaccgtctcctcagcttccaccaagggcccatccgtcttccccctggcgccctgctctagaagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgatag

SEQ ID NO: 64 - protein, 471 aa, Homo sapiens
MELGLRWVFLVAILEGVQCEVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARDGYSSGWSYYYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 65 - DNA, 723 bp, Homo sapiens
atgaggctccctgctcagctcctggggctgctaatgctctggatacctggatccagtgcagatattgtgatgacccagactccactctctctgtccgtcactcctggacagccggcctccatctcctgcaagtctagtcagagcctcctgcttagtgatggaaagacctatttgaattggtacctgcagaagcccggccagcctccacagctcctgatctatgaagtttccaaccggttctctggagtgccagacaggttcagtggcagcgggtcagggacagatttcacactgaaaatcagccgggtggaggctgaggatgttggggtttattactgcatgcaaagtatacagcttatgtgcagttttggccaggggaccaagctggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagtga

SEQ ID NO: 66 - protein, 239 aa, Homo sapiens
MRLPAQLLGLLMLWIPGSSADIVMTQTPLSLSVTPGQPASISCKSSQSLLLSDGKTYLNWYLQKPGQPPQLLIYEVSNRFSGVPDRFSGSGSGTDFTLKISRVEAEDVGVYSCMQSIQLMSSFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 67 - DNA, 723 bp, Homo sapiens
atgaggctccctgctcagctcctggggctgctaatgctctggatacctggatccagtgcggatattgtgatgacccagactccactctctctgtccgtcacccctggacagccggcctccatctcctgcaagtctagtcagagcctcctgtatagtgatggaaagacctatttgttttggtacctgcagaagccaggccagcctccacagctcctgatctatgaagtttccaaccgattctctggagtgccagataggttcagtggcagcgggtcagggacagatttcacactgaaaatcagccgggtggaggctgaggatgttggggtttattactgcatgcaaagtatacagcttccgtggacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagtga

SEQ ID NO: 68 - protein, 239 aa, Homo sapiens
MRLPAQLLGLLMLWIPGSSADIVMTQTPLSLSVTPGQPASISCKSSQSLLYSDGKTYLFWYLQKPGQPPQLLIYEVSNRFSGVPDRFSGSGSGTDFTLKISRVEAEDVGVYYCMQSIQLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC